(19) United States
(12) Patent Application Publication
(10) Pub. No.: US 2009/0227600 A1
(43) Pub. Date: Sep. 10, 2009

(54) 4-(PYRAZINE-2-YL) -PYRROLIDINE -2-CARBOXYLIC ACID COMPOUNDS AND DERIVATIVES THEREOF AS HEPATITIS C VIRUS INHIBITORS

(76) Inventors: Rossella Guidetti, Hertfordshire (GB); David Haigh, Hertfordshire (GB); Charles David Hartley, Hertfordshire (GB); Peter David Howes, Hertfordshire (GB); Fabrizio Nerozzi, Hertfordshire (GB); Stephen Allan Smith, Hertfordshire (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 11/577,940

(22) PCT Filed: Oct. 24, 2005
(86) PCT No.: PCT/EP05/11542

§ 371 Date: Apr. 25, 2007

(30) Foreign Application Priority Data
Oct. 25, 2004 (GB) 0423672.5

(51) Int. Cl. A61K 31/497; C07D 417/14; A61P 31/12

(52) U.S. Cl. 514/255.05; 544/405

(57) ABSTRACT

Anti-viral agents of Formula (Ia)

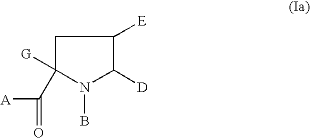

wherein:
A represents hydroxy; B represents C(O)R³ wherein R³ is 4-tert-butyl-3-methoxyphenyl; D represents 1,3-thiazol-2-yl or 5-methyl-1,3-thiazol-2-yl; E represents pyrazin-2-yl; G represents 1,3-thiazol-2-ylmethyl, 1,3-thiazol-4-ylmethyl, 1,2-thiazol-3-ylmethyl, or 1H-pyrazol-1-ylmethyl; and salts, solvates and esters thereof; provided that when A is esterified to form —OR where R is selected from straight or branched chain alkyl, aralkyl, aryloxyalkyl, or aryl, then R is other than tert-butyl, processes for their preparation and their use in HCV treatment are provided.

### FIELD OF THE INVENTION

The present invention relates to novel C(4)-pyrazine acyl pyrrolidine derivatives useful as anti-viral agents. Specifically, the present invention involves novel Hepatitis C Virus (HCV) inhibitors.

### BACKGROUND OF THE INVENTION

Infection with HCV is a major cause of human liver disease throughout the world. In the US, an estimated 4.5 million Americans are chronically infected with HCV. Although only 30% of acute infections are symptomatic, greater than 85% of infected individuals develop chronic, persistent infection. Treatment costs for HCV infection have been estimated at $5.46 billion for the US in 1997. Worldwide over 200 million people are estimated to be infected chronically. HCV infection is responsible for 40-60% of all chronic liver disease and 30% of all liver transplants. Chronic HCV infection accounts for 30% of all cirrhosis, end-stage liver disease, and liver cancer in the U.S. The CDC estimates that the number of deaths due to HCV will minimally increase to 38,000/year by the year 2010.

Due to the high degree of variability in the viral surface antigens, existence of multiple viral genotypes, and demonstrated specificity of immunity, the development of a successful vaccine in the near future is unlikely. Alpha-interferon (alone or in combination with ribavirin) has been widely used since its approval for treatment of chronic HCV infection. However, adverse side effects are commonly associated with this treatment: flu-like symptoms, leukopenia, thrombocytopenia, depression from interferon, as well as anemia induced by ribavirin (Lindsay, K. L. (1997) Hepatology 26 (suppl 1): 71S-77S). This therapy remains less effective against infections caused by HCV genotype 1 (which constitutes ˜75% of all HCV infections in the developed markets) compared to infections caused by the other 5 major HCV genotypes. Unfortunately, only ˜50-80% of the patients respond to this treatment (measured by a reduction in serum HCV RNA levels and normalization of liver enzymes) and, of those treated, 50-70% relapse within 6 months of cessation of treatment. Recently, with the introduction of pegylated interferon, both initial and sustained response rates have improved substantially, and combination treatment of Peg-IFN with ribavirin constitutes the gold standard for therapy. However, the side effects associated with combination therapy and the impaired response in patients with genotype 1 present opportunities for improvement in the management of this disease.

First identified by molecular cloning in 1989 (Choo, Q-L et al (1989) Science 244:359-362), hepatitis C virus (HCV) is now widely accepted as the most common causative agent of post-transfusion non A, non-B hepatitis (NANBH) (Kuo, G et al (1989) Science 244:362-364). Due to its genome structure and sequence homology, this virus was assigned as a new genus in the Flaviviridae family. Like the other members of the Flaviviridae, such as flaviviruses (e.g. yellow fever virus and Dengue virus types 1-4) and pestiviruses (e.g. bovine viral diarrhea virus, border disease virus, and classic swine fever virus) (Choo, Q-L et al (1989) Science 244:359-3; Miller, R. H. and R. H. Purcell (1990) Proc. Natl. Acad. Sci. USA 87:2057-2061), HCV is an enveloped virus containing a single strand RNA molecule of positive polarity. The HCV genome is approximately 9.6 kilobases (kb) with a long, highly conserved, noncapped 5′ nontranslated region (NTR) of approximately 340 bases which functions as an internal ribosome entry site (IRES) (Wang C Y et al ‘An RNA pseudoknot is an essential structural element of the internal ribosome entry site located within the hepatitis C virus 5′ noncoding region’ RNA—A Publication of the RNA Society. 1(5): 526-537, 1995 Jul.). This element is followed by a region which encodes a single long open reading frame (ORF) encoding a polypeptide of −3000 amino acids comprising both the structural and nonstructural viral proteins.

Upon entry into the cytoplasm of the cell, this RNA is d erectly translated into a polypeptide of ˜3000 amino acids comprising both the structural and nonstructural viral proteins. This large polypeptide is subsequently processed into the individual structural and nonstructural proteins by a combination of host and virally-encoded proteinases (Rice, C. M. (1996) in B. N. Fields, D. M. Knipe and P. M. Howley (eds) Virology 2^{nd} Edition, p 931-960; Raven Press, N.Y.). Following the termination codon at the end of the long ORF, there is a 3′ NTR which roughly consists of three regions: an ˜40 base region which is poorly conserved among various genotypes, a variable length poly(U)/polypyrimidine tract, and a highly conserved 98 base element also called the “3′ X-tail” (Kolykhalov, A. et al (1996) J. Virology 70:3363-3371; Tanaka, T. et al (1995) Biochem Biophys. Res. Commun. 215:744-749; Tanaka, T. et al (1996) J. Virology 70:3307-3312; Yamada, N. et al (1996) Virology 223:255-261.). The 3′ NTR is predicted to form a stable secondary structure which is essential for HCV growth in chimps and is believed to function in the initiation and regulation of viral RNA replication.

The NS5B protein (591 amino acids, 65 kDa) of HCV (Behrens, S. E. et al (1996) EMBO J. 15:12-22), encodes an RNA-dependent RNA polymerase (RdRp) activity and contains canonical motifs present in other RNA viral polymerases. The NS5B protein is fairly well conserved both intra-typically (˜95-98% amino acid (aa) identity across 1b isolates) and inter-typically (˜85% aa identity between genotype 1a and 1b isolates). The essentiality of the HCV NS5B RdRp activity for the generation of infectious progeny virions has been formally proven in chimpanzees (A. A. Kolykhalov et al. (2000) Journal of Virology, 74(4), p. 2046-2051). Thus, inhibition of NS5B RdRp activity (inhibition of RNA replication) is predicted to cure HCV infection.

Although the predominant HCV genotype worldwide is genotype 1, this itself has two main subtypes, denoted 1a and 1a. As seen from entries into the Los Alamos HCV database (www.hcv.lanl.gov) (Table 1) there are regional differences in the distribution of these subtypes: while genotype 1a is most abundant in the United States, the majority of sequences in Europe and Japan are from genotype 1b.

TABLE 1

| % of sequences | | | | |
|---|---|---|---|---|
| in the database | World | USA | Europe | Japan |
| Genotype 1 | 71.8 | 87.8 | 75.9 | 80.2 |
| Genotype 1a | 28.4 | 66.4 | 21.7 | 1.6 |
| Genotype 1b | 43.4 | 21.4 | 54.2 | 78.6 |

The prevalance of genotype 1a in some regions makes it highly desirable to identify an anti-viral agent that is able to inhibit both genotype 1a and genotype 1b. This means a wider patient pool would be able to benefit from treatment with the same agent.

Based on the foregoing, there exists a significant need to identify synthetic or biological compounds for their ability to inhibit replication of both genotype 1a and genotype 1b of HCV.

PCT publication number WO2003/037895 generically discloses certain compounds, including certain acyl pyrrolidine compounds, having HCV inhibitory activity against the 1a genotype. The compounds disclosed have the formula (I)

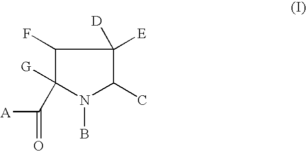

wherein:
A represents OR¹, NR¹R², or R¹ wherein R¹ and R² are independently selected from the group consisting of hydrogen, C₁₋₆alkyl, aryl, heteroaryl, arylalkyl, and heteroarylalkyl; or
R¹ and R² together with the nitrogen atom to which they are attached form a 5 or 6 membered saturated cyclic group;
B represents C(O)R³ wherein R³ is selected from the group consisting of C₁₋₆alkyl, aryl, heteroaryl, arylalkyl, and heteroarylalkyl;
C represents C₁₋₆alkyl, aryl, heteroaryl or heterocyclyl;
D represents a saturated or unsaturated 6-membered heterocyclic ring comprising three or more carbon atoms, each of which may independently be optionally substituted by R⁴ and R⁵, and one to three heteroatoms independently selected from N, optionally substituted by hydrogen, C₁₋₆alkyl, C(O)R³, SO₂R³, aryl, heteroaryl, arylalkyl, or heteroarylalkyl; O; and S, optionally substituted by one or two oxygen atoms; wherein the 6 membered ring may be attached at any endocyclic carbon atom, and may be optionally fused to a saturated or unsaturated 5 or 6 membered carbocyclic or heterocyclic ring which may itself be optionally substituted on a non-fused carbon atom by C₁₋₆alkyl, halo, OR⁸, C(O)NR⁶R⁷, C(O)R³, CO₂H, CO₂R³, NR⁶R⁷, NHC(O)R³, NHCO₂R³, NHC(O)NR¹R², SO₂NR¹R², SO₂R³, nitro, cyano, oxo, aryl, heteroaryl and heterocyclyl;
R⁴ and R⁵ are independently selected from hydrogen, C₁₋₆alkyl, halo, ORB, C(O)NR⁶R⁷, C(O)R³, CO₂H, CO₂R³, NR⁶R⁷, NHC(O)R³, NHCO₂R³, NHC(O)NR¹R², SO₂NR¹R², SO₂R³, nitro, oxo, aryl, heteroaryl and heterocyclyl;
R⁶ and R⁷ are independently selected from hydrogen, C₁₋₆alkyl, aryl and heteroaryl; and
R⁸ represents hydrogen, C₁₋₆alkyl, arylalkyl, or heteroarylalkyl;
E represents hydrogen or C₁₋₆alkyl;
F represents hydrogen, C₁₋₆alkyl, aryl or heteroaryl; and
G represents hydrogen, C₁₋₆alkyl, heterocyclylalkyl, arylalkyl or heteroarylalkyl; and salts, solvates and esters thereof, provided that when A is OR¹ then R¹ is other than tert-butyl.

Surprisingly, it has now been found that compounds according to the present invention, generically disclosed in WO2003/037895, and having a specific substitution pattern, exhibit improved properties over those compounds specifically disclosed in WO2003/037895.

### SUMMARY OF THE INVENTION

The present invention involves C(2)-heteroarylmethyl-C(4)-pyrazin-2-yl acyl pyrrolidine compounds represented hereinbelow, pharmaceutical compositions comprising such compounds and use of the compounds in treating viral infection, especially HCV infection.

### DETAILED DESCRIPTION OF THE INVENTION

The present invention provides at least one chemical entity chosen from compounds of Formula (Ia):

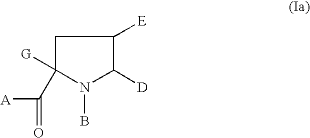

wherein:
A represents hydroxy;
B represents C(O)R³ wherein R³ is 4-tert-butyl-3-methoxyphenyl;
D represents 1,3-thiazol-2-yl or 5-methyl-1,3-thiazol-2-yl;
E represents pyrazin-2-yl;
G represents 1,3-thiazol-2-ylmethyl, 1,3-thiazol-4-ylmethyl, 1,2-thiazol-3-ylmethyl, or 1H-pyrazol-1-ylmethyl;
and salts, solvates and esters thereof; provided that when A is esterified to form —OR where R is selected from straight or branched chain alkyl, aralkyl, aryloxyalkyl, or aryl, then R is other than tert-butyl.

In one aspect, the relative stereochemistry of racemic compounds of Formula (Ia), is represented by Formulae (Ip) or (Iq):

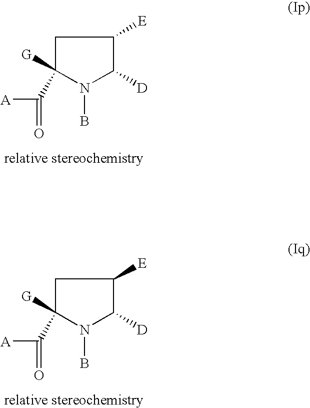

wherein A, B, D, E and G are as defined above for Formula (Ia).

In a further aspect, the absolute stereochemistry of chiral compounds of Formula (Ia) is represented by Formulae (Ipp) or (Iqq):

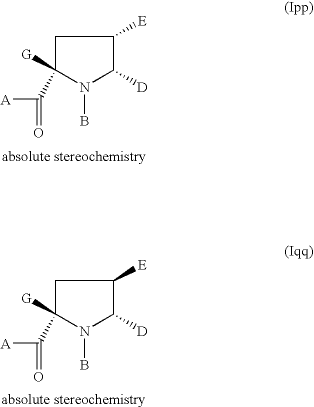

wherein A, B, D, E and G are as defined above for Formula (Ia).

The following substituent groups are preferred, where applicable, in respect of each of Formulae Ia, Ip, Ipp Iq and Iqq:

In one aspect, A is hydroxy (that is, not esterified).

In one aspect, G represents 1,3-thiazol-4-ylmethyl or 1H-pyrazol-1-ylmethyl. In a further aspect, G represents 1,3-thiazol-4-ylmethyl. In another aspect, G represents 1,3-thiazol-4-ylmethyl and D represents 1,3-thiazol-2-yl.

In one aspect, the compounds of Formula (Ia) are represented by compounds of Formula (Ipp).

It is to be understood that the present invention covers all combinations of aspects, suitable, convenient and preferred groups described herein.

The chemical entities of the present invention exhibit an improved genotype-1a/1a profile against HCV polymerase, and therefore have the potential to achieve efficacy in man over a broad patient population.

The term ‘genotype-1a/1a profile’ means potency as an inhibitor of HCV polymerase enzyme in wildtype HCV of the 1a genotype and of the 1a genotype. High potency in both genotypes is considered to be advantageous.

There is provided as a further aspect of the present invention at least one chemical entity chosen from compounds of Formula (Ia) and physiologically acceptable salts, solvates or esters thereof for use in human or veterinary medical therapy, particularly in the treatment or prophylaxis of viral infection, particularly HCV infection.

It will be appreciated that reference herein to therapy and/or treatment includes, but is not limited to prevention, retardation, prophylaxis, therapy and cure of the disease. It will further be appreciated that references herein to treatment or prophylaxis of HCV infection includes treatment or prophylaxis of HCV-associated disease such as liver fibrosis, cirrhosis and hepatocellular carcinoma.

According to another aspect of the invention, there is provided the use of at least one chemical entity chosen from compounds of Formula (Ia) and physiologically acceptable salts, solvates or esters thereof in the manufacture of a medicament for the treatment and/or prophylaxis of viral infection, particularly HCV infection.

In a further or alternative aspect there is provided a method for the treatment of a human or animal subject with viral infection, particularly HCV infection, which method comprises administering to said human or animal subject an effective amount of at least one chemical entity chosen from compounds of Formula (Ia) and physiologically acceptable salts, solvates or esters thereof.

It will be appreciated that the compounds of the present invention may contain one or more asymmetric carbon atoms and may exist in racemic, diastereoisomeric, and optically active forms.

In one aspect, chemical entities useful in the present invention may be chosen from compounds of Formula (Ia) selected from the group consisting of:
rel-(2R,4S,5R)-1-(3-Methoxy-4-tert-butylbenzoyl)-4-(pyrazin-2-yl)-2-(1,3-thiazol-4-ylmethyl)-5-(1,3-thiazol-2-yl)pyrrolidine-2-carboxylic acid;
rel-(2R,4S,5R)-1-(3-Methoxy-4-tert-butylbenzoyl)-4-(pyrazin-2-yl)-2-(1,3-thiazol-2-ylmethyl)-5-(1,3-thiazol-2-yl)pyrrolidine-2-carboxylic acid;
rel-(2R,4S,5R)-1-(3-Methoxy-4-tert-butylbenzoyl)-5-(5-methyl-1,3-thiazol-2-yl)-4-(pyrazin-2-yl)-2-(1,2-thiazol-3-ylmethyl)pyrrolidine-2-carboxylic acid;
rel-(2R,4S,5R)-1-(3-Methoxy-4-tert-butylbenzoyl)-4-(pyrazin-2-yl)-2-(1,2-thiazol-3-ylmethyl)-5-(1,3-thiazol-2-yl)pyrrolidine-2-carboxylic acid;
rel-(2R,4S,5R)-1-(3-Methoxy-4-tert-butylbenzoyl)-5-(5-methyl-1,3-thiazol-2-yl)-4-(pyrazin-2-yl)-2-(1H-pyrazol-1-ylmethyl)pyrrolidine-2-carboxylic acid;
rel-(2R,4S,5R)-1-(3-Methoxy-4-tert-butylbenzoyl)-4-(pyrazin-2-yl)-2-(1H-pyrazol-1-ylmethyl)-5-(1,3-thiazol-2-yl)pyrrolidine-2-carboxylic acid;
rel-(2R,4S,5R)-1-(3-Methoxy-4-tert-butylbenzoyl)-5-(5-methyl-1,3-thiazol-2-yl)-4-(pyrazin-2-yl)-2-(1,3-thiazol-2-ylmethyl)pyrrolidine-2-carboxylic acid; and
rel-(2R,4S,5R)-1-(3-Methoxy-4-tert-butylbenzoyl)-5-(5-methyl-1,3-thiazol-2-yl)-4-(pyrazin-2-yl)-2-(1,3-thiazol-4-ylmethyl)pyrrolidine-2-carboxylic acid;
   and salts, solvates and esters, and individual enantiomers thereof.

In a further aspect, chemical entities useful in the present invention may be chosen from compounds of Formula (Ia) selected from the group consisting of:
rel-(2R,4S,5R)-1-(3-Methoxy-4-tert-butylbenzoyl)-4-(pyrazin-2-yl)-2-(1,3-thiazol-4-ylmethyl)-5-(1,3-thiazol-2-yl)pyrrolidine-2-carboxylic acid;
rel-(2R,4S,5R)-1-(3-Methoxy-4-tert-butylbenzoyl)-5-(5-methyl-1,3-thiazol-2-yl)-4-(pyrazin-2-yl)-2-(1H-pyrazol-1-ylmethyl)pyrrolidine-2-carboxylic acid;
rel-(2R,4S,5R)-1-(3-Methoxy-4-tert-butylbenzoyl)-4-(pyrazin-2-yl)-2-(1H-pyrazol-1-ylmethyl)-5-(1,3-thiazol-2-yl)pyrrolidine-2-carboxylic acid;
rel-(2R,4S,5R)-1-(3-Methoxy-4-tert-butylbenzoyl)-5-(5-methyl-1,3-thiazol-2-yl)-4-(pyrazin-2-yl)-2-(1,3-thiazol-4-ylmethyl)pyrrolidine-2-carboxylic acid;
   and salts, solvates and esters, and individual enantiomers thereof.

In a yet further aspect, chemical entities useful in the present invention may be chosen from compounds of Formula (Ia) selected from the group consisting of:
rel-(2R,4S,5R)-1-(3-Methoxy-4-tert-butylbenzoyl)-4-(pyrazin-2-yl)-2-(1,3-thiazol-4-ylmethyl)-5-(1,3-thiazol-2-yl)pyrrolidine-2-carboxylic acid;
rel-(2R,4S,5R)-1-(3-Methoxy-4-tert-butylbenzoyl)-5-(5-methyl-1,3-thiazol-2-yl)-4-(pyrazin-2-yl)-2-(1,3-thiazol-4-ylmethyl)pyrrolidine-2-carboxylic acid;
   and salts, solvates and esters, and individual enantiomers thereof.

In another aspect, chemical entities useful in the present invention may be chosen from compounds of Formula (Ia) selected from the group consisting of:
rel-(2R,4S,5R)-1-(3-Methoxy-4-tert-butylbenzoyl)-4-(pyrazin-2-yl)-2-(1,3-thiazol-4-ylmethyl)-5-(1,3-thiazol-2-yl)pyrrolidine-2-carboxylic acid;
   and salts, solvates and esters, and individual enantiomers thereof.

Also included in the present invention are pharmaceutically acceptable salt complexes. The present invention also covers the physiologically acceptable salts of the compounds of formula (Ia). Suitable physiologically acceptable salts of the compounds of formula (Ia) include acid salts, for example sodium, potassium, calcium, magnesium and tetraalkylammonium and the like, or mono- or di-basic salts with the appropriate acid for example organic carboxylic acids such as acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids and the like.

The present invention also relates to solvates of the compounds of Formula (Ia), for example hydrates.

The present invention also relates to pharmaceutically acceptable esters of the compounds of Formula (Ia), for example carboxylic acid esters —COOR, in which R is selected from straight or branched chain alkyl, for example n-propyl, n-butyl, alkoxyalkyl (e.g. methoxymethyl), alkoxycarbonylalkyl (e.g. methoxycarbonylmethyl), acyloxyalkyl (e.g. pivaloyloxymethyl), aralkyl (e.g. benzyl), aryloxyalkyl (e.g. phenoxymethyl), aryl (e.g. phenyl optionally substituted by halogen, C₁₋₄alkyl or C₁₋₄alkoxy or amino). Unless otherwise specified, any alkyl moiety present in such esters preferably contains 1 to 18 carbon atoms, particularly 1 to 4 carbon atoms. Any aryl moiety present in such esters preferably comprises a phenyl group.

In one aspect, the compound of Formula (Ia) is in the form of the parent compound, a salt or a solvate.

As used herein, the term “pharmaceutically acceptable” used in relation to an ingredient (active ingredient such as an active ingredient, a salt thereof or an excipient) which may be included in a pharmaceutical formulation for administration to a patient, refers to that ingredient being acceptable in the sense of being compatible with any other ingredients present in the pharmaceutical formulation and not being deleterious to the recipient thereof.

It will further be appreciated that certain compounds of the present invention may exist in different tautomeric forms. All tautomers are contemplated to be within the scope of the present invention.

Compounds of Formula (Ia) in which A is hydroxy may be prepared from a compound of Formula (II)

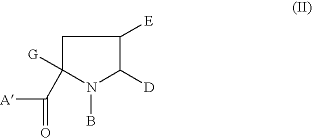

in which A′ is a protected hydroxy group, for example an alkoxy, benzyloxy or silyloxy, for example tri-(C₁₋₄alkyl)-silyloxy group, and B, D, E and G are as defined above for Formula (Ia), by deprotection. Suitable protecting groups can be found, but are not restricted to, those found in T W Greene and P G M Wuts ‘Protective Groups in Organic Synthesis’, 3^{rd} Ed (1999), J Wiley and Sons.

For example when A′ is tert-butoxy, and B, D, E and G are as defined above for Formula (Ia), by treatment with an appropriate acid, for example trifluoroacetic acid. Optionally, the reaction is carried out in a solvent, for example dichloromethane. Preferably, the temperature is in the range 0 to 50° C., more preferably 20 to 30° C.

For example when A′ is benzyloxy, and B, D, E and G are as defined above for Formula (Ia), by hydrogenolysis in the presence of a suitable catalyst for example palladium-on-carbon. Suitably, the reaction is carried out in a solvent, for example ethanol. Preferably, the temperature is in the range 0 to 50° C.

For example when A′ is allyloxy, and B, D, E and G are as defined above for Formula (Ia), by treatment with a suitable catalyst for example tetrakis(triphenylphosphine)palladium(0) and a suitable proton source, for example phenylsilane. The reaction is carried out in a suitable solvent, for example dichloromethane.

For example when A′ is tri(methyl)silyloxy, and B, D, E and G are as defined above for Formula (Ia), by treatment with a suitable fluoride source for example tetrabutylammonium fluoride. The reaction is carried out in a suitable solvent, for example tetrahydrofuran.

Compounds of Formula (Ia) or (II) may be prepared by reaction of a compound of Formula (III)

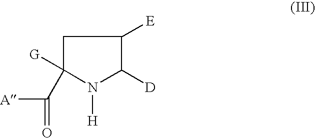

in which A″ is hydroxy or an alkoxy, benzyloxy or tri-(C₁₋₄alkyl)-silyloxy group, and D, E and G are as defined above for Formula (Ia); with a suitable acylating agent, for example R³—C(O)-hal, wherein hal is a halo atom, preferably chloro or bromo, and R³ is as defined above for Formula (Ia). Preferably the reaction is carried out in a suitable solvent, for example dichloromethane, in the presence of a suitable base, for example triethylamine. Preferably, the temperature is in the range 0 to 50° C., more preferably 20 to 30° C. Optionally, the reaction may be carried out at the reflux temperature of the solvent.

Compounds of Formula (III) may be prepared by reaction of a compound of Formula (IV)

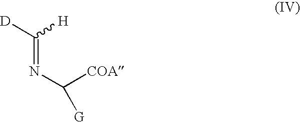

in which D and G are as defined above for Formula (Ia) and A″ is as defined above for Formula (III) with a compound of Formula (V)

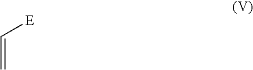

in which E is as defined above for Formula (Ia). Preferably, the reaction is carried out in a suitable solvent, for example THF or acetonitrile, optionally in the presence of a Lewis acid catalyst, such as lithium bromide or silver acetate, and a base, such as triethylamine, 1,8-diazabicyclo[5,4,0]undec-7-ene (DBU) or tetramethyl guanidine. Preferably, the temperature is in the range 0 to 50° C., more preferably 20 to 30° C. Alternatively, the reaction is carried out in a suitable solvent, for example THF or acetonitrile, in the presence of an acid, such as acetic acid, or the reaction may be carried out by heating compounds of Formula (IV) and Formula (V) in a suitable solvent, for example toluene, xylene or acetonitrile in the absence of a catalyst.

Compounds of Formula (III) may also be prepared in a one pot synthesis by reaction of a compound of Formula (VI)

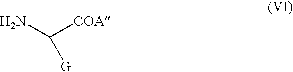

in which G is as defined above for Formula (Ia) and A″ is as defined above for Formula (III), with a compound of Formula (V) and a compound of Formula D-CHO. Preferably, the reaction is carried out in a suitable solvent, for example THF or acetonitrile, optionally in the presence of a Lewis acid catalyst, such as lithium bromide or silver acetate, and a base, such as triethylamine, 1,8-diazabicyclo[5,4,0]undec-7-ene (DBU) or tetramethyl guanidine. Preferably the reaction is carried out at a temperature in the range 0 to 50° C., more preferably 20 to 30° C. Optionally a drying agent is used in the process, for example molecular sieves.

Compounds of Formula (IV) may be prepared by reaction of a compound of Formula (VI) in which G is as defined above for Formula (Ia) and A″ is as defined above for Formula (III) with a compound of Formula D-CHO in which D is as defined above for Formula (Ia) optionally in the presence of a suitable drying agent, for example magnesium sulphate, in a suitable solvent, for example dichloromethane. Preferably the reaction is carried out at a temperature in the range 0 to 50° C.

Compounds of Formula (VI) in which G is 1H-pyrazol-1-ylmethyl, and A″ is hydroxy, may be prepared by treatment of a compound of (VII)

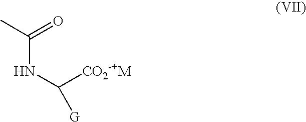

in which G is 1H-pyrazol-1-ylmethyl and M is a metal cation, for example potassium, with a suitable acid, for example 10% aqueous hydrochloric acid, in the presence of an ion exchange resin, such as Amberlyst™ 120 (H⁺).

Compounds of Formula (VI) in which G is 1,3-thiazol-2-ylmethyl or 1H-pyrazol-1-ylmethyl, and A″ is an alkoxy, benzyloxy or tri-(C₁₋₄alkyl)-silyloxy group, may be prepared by treatment of a compound of Formula (VI) in which G is 1,3-thiazol-2-ylmethyl or 1H-pyrazol-1-ylmethyl, and A″ is hydroxy, by conventional esterification or protecting group procedures. For example, a compound of Formula (VI) in which G is 1,3-thiazol-2-ylmethyl or 1H-pyrazol-1-ylmethyl, and A″ is tert-butoxy may be prepared by treatment of a compound of Formula (VI) in which G is 1,3-thiazol-2-ylmethyl or 1H-pyrazol-1-ylmethyl, and A″ is hydroxy, with an appropriate tert-butyl transfer agent, such as tert-butylacetate in the presence of a suitable acid catalyst, such as 70% perchloric acid.

Compounds of Formula (VI) in which G is 1,3-thiazol-2-ylmethyl or 1H-pyrazol-1-ylmethyl, and A″ is hydroxy are known in the art.

Compounds of Formula (VII) in which G is 1H-pyrazol-1-ylmethyl, may additionally be prepared by reaction of a compound of Formula (VIII)

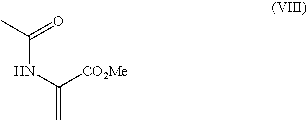

with 1H-pyrazole, in the presence of a suitable base, for example potassium carbonate when M is potassium, and in the presence of a suitable solvent, such as aqueous acetonitrile. Preferably the reaction is carried out at a temperature in the range 50-70° C., more preferably 60° C.

Compounds of Formula (VI) in which G is 1,3-thiazol-2-ylmethyl, 1,3-thiazol-4-ylmethyl or 1,2-thiazol-3-ylmethyl and A″ is an alkoxy, benzyloxy or tri-(C₁₋₄alkyl)-silyloxy group, may be prepared by treatment of a compound of Formula (IX)

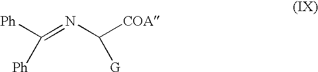

in which G is 1,3-thiazol-2-ylmethyl, 1,3-thiazol-4-ylmethyl or 1,2-thiazol-3-ylmethyl and A″ is an alkoxy, benzyloxy or tri-(C₁₋₄alkyl)-silyloxy group with an acid, for example 15% aqueous citric acid. Preferably, the reaction is carried out in a suitable solvent, for example THF.

Compounds of Formula (IX) may be prepared by reaction of a compound of Formula (X)

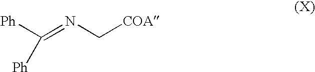

in which A″ is an alkoxy, benzyloxy or tri-(C₁₋₄alkyl)-silyloxy group with a compound of Formula G-hal in which G is 1,3-thiazol-2-ylmethyl, 1,3-thiazol-4-ylmethyl or 1,2-thiazol-3-ylmethyl, and hal is a halo atom, preferably chloro or bromo. In one aspect, the reaction is carried out in the presence of a suitable base, such as potassium t-butoxide. In one aspect, the reaction is carried out in a suitable solvent, for example THF. In one aspect, the reaction is carried out in the presence of a suitable catalyst, for example lithium iodide. In one aspect, the reaction is carried out at a temperature in the range −10° C. to room temperature, suitably 0° C.

Compounds of Formula (Ia) or (II) may also be prepared by reaction of a compound of Formula (XI)

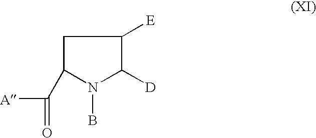

in which A″ is hydroxy or an alkoxy, benzyloxy or tri-(C₁₋₄alkyl)-silyloxy group, and B, D and E are as defined above for Formula (Ia); with a compound of Formula G-hal in which G is as defined above for Formula (I) and hal is a halo atom, preferably chloro or bromo. In one aspect, the reaction is carried out in a suitable solvent, for example THF, in the presence of a suitable base, for example lithium hexamethyldisilazide (LHMDS).

Compounds of Formula (XI) may be prepared by reaction of a compound of Formula (XII)

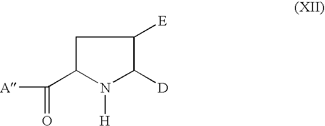

in which A″, D and E are as defined above for Formula (Ia), with a suitable acylating agent, for example R³—C(O)-hal, wherein hal is a halo atom, preferably chloro or bromo, and R³ is as defined above for Formula (Ia). Preferably the reaction is carried out in a suitable solvent, for example dichloromethane, in the presence of a suitable base, for example triethylamine. Preferably, the temperature is in the range 0 to 50° C., suitably 20 to 30° C. Optionally, the reaction may be carried out at the reflux temperature of the solvent.

Compounds of Formula (XII) may be prepared in a one pot synthesis by reaction of a compound of Formula (XIII)

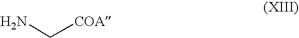

in which A″ is as defined above for Formula (III), with a compound of Formula (V) and a compound of Formula D-CHO. Preferably, the reaction is carried out in a suitable solvent, for example THF or acetonitrile, optionally in the presence of a Lewis acid catalyst, such as lithium bromide or silver acetate, and a base, such as triethylamine, 1,8-diazabicyclo[5,4,0]undec-7-ene (DBU) or tetramethyl guanidine. Preferably the reaction is carried out at a temperature in the range 0 to 50° C., suitably 20 to 30° C. Optionally a drying agent is used in the process, for example molecular sieves.

Compounds of Formula (V), (VIII), (X), (XIII), G-hal and D-CHO are known in the art or may be prepared by standard literature procedures.

It will be appreciated that the present invention provides a method for the interconversion of the rel-(2R,4S,5R)-diastereoisomer of a compound of formula (I) or (II) wherein A is other than hydroxy, into the rel-(2R,4R,5R)-diastereoisomer. For example base-catalysed epimerisation by treatment of the rel-(2R,4S,5R)-diastereoisomer with a suitable base, such as aqueous sodium hydroxide, in the presence of a suitable solvent, such as methanol.

Compounds of Formula (Ia) in which A is an ester may be prepared by esterification of compounds of Formula (Ia) in which A is hydroxy by standard literature procedures for esterification.

It will be appreciated that compounds of Formula (Ia), (II), (III), (XI) and (XII) which exist as diastereoisomers may optionally be separated by techniques well known ink the art, for example by column chromatography.

It will be appreciated that racemic compounds of Formula (Ia), (II), (III), (XI) and (XII) may be optionally resolved into their individual enantiomers. Such resolutions may conveniently be accomplished by standard methods known in the art. For example, a racemic compound of Formula (Ia), (II), (III), (XI) and (XII) may be resolved by chiral preparative HPLC. Alternatively, racemic compounds of Formula (Ia), (II), (III), (XI) and (XII) which contain an appropriate acidic or basic group, such as a carboxylic acid group or amine group may be resolved by standard diastereoisomeric salt formation with a chiral base or acid reagent respectively as appropriate. Such techniques are well established in the art. For example, a racemic compound of Formula (III) may be resolved by treatment with a chiral acid such as (R)-(−)-1,1′-binaphthyl-2,2′-diyl-hydrogen phosphate, in a suitable solvent, for example dichloromethane, isopropanol or acetonitrile. The enantiomer of Formula (III) may then be obtained by treating the salt with a suitable base, for example triethylamine, in a suitable solvent, for example methyl tert-butyl ether. Individual enantiomers of Formula (II) and/or (III) may then be progressed to an enantiomeric compound of Formula (Ia) by the chemistry described above in respect of racemic compounds.

It will also be appreciated that Individual enantiomeric compounds of Formula (III) and/or (XII) may be prepared by general methods of asymmetric synthesis using, where appropriate, chiral auxiliaries or chiral catalytic reagents and additionally performing any suitable functional group interconversion step as hereinbefore described, including the addition or removal of any such chiral auxiliary. Such general methods of asymmetric synthesis are well known in the art and include, but are not restricted to, those described in “Asymmetric Synthesis,” Academic Press, 1984 and/or “Chiral Auxiliaries and Ligands in Asymmetric Synthesis”, Wiley, 1995. For example, suitable general chiral auxiliaries include chiral alcohols such as menthol or 1-phenylethanol; chiral oxazolidinones such as 4-benzyloxazolidin-2-one or 4-isopropyloxazolidin-2-one; chiral sultams such as camphor sultam; or chiral amines such as 1-phenylethylamine or 2-amino-2-phenylethanol. Suitable general chiral catalytic reagents include chiral basic amines and chiral ligands such as N-methylephedrine, 1-phenyl-2-(1-pyrrolidinyl)-1-propanol, 3-(dimethylamino)-1,7,7-trimethylbicyclo[2.2.1]-heptan-2-ol, 3,4-bis(diphenylphosphanyl)-1-(phenylmethyl)-pyrrolidine, chinchonine, chinchonidine, sparteine, hydroquinine or quinine, BINAP or chiral bis(oxazoline) (BOX) ligands and derivatives, optionally in the presence of a metal salt, for example MₘXₓ where M is silver, cobalt, zinc, titanium, magnesium, or manganese, and X is halide (for example chloride or bromide), acetate, trifluoroacetate, p-toluenesulfonate, trifluoromethylsulfonate, hexafluorophosphate or nitrate, and ₘ and ₓ are 1, 2, 3 or 4, and optionally in the presence of a base, for example triethylamine. All of these chiral auxiliaries or chiral catalytic reagents are well described in the art. General illustrative examples of the preparation of various chiral pyrrolidines by asymmetric synthesis using chiral auxiliaries or chiral catalytic reagents include, but are not limited to, those described in Angew. Chem. Int Ed., (2002), 41: 4236; Chem. Rev., (1998), 98: 863; J. Am. Chem. Soc., (2002), 124: 13400; J. Am. Chem. Soc., (2003), 125: 10175; Org. Lett., (2003), 5, 5043; Tetrahedron, (1995), 51: 273; Tetrahedron: Asymm., (1995), 6: 2475; Tetrahedron: Asymm., (2001), 12: 1977; Tetrahedron: Asymm., (2002), 13: 2099 and Tet Lett., (1991), 41: 5817.

In a particular aspect, a chiral pyrrolidine compound of Formula (IIIa)

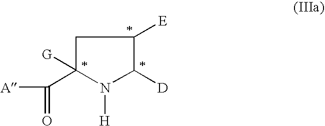

in which A″, D, E, and G are as defined above for Formula (III), and * denotes an enantioenriched chiral centre can be prepared by reaction of a compound of Formula (IV) with a compound of Formula (V) as herein before defined, under asymmetric reaction conditions. It will be appreciated by those skilled in the art that such asymmetric reaction conditions may be afforded by, for example, the inclusion in the reaction mixture of a chiral catalytic reagent as herein before defined.

In one aspect, the reaction is carried out in the presence of a suitable chiral catalytic reagent, for example (−)-N-methylephedrine, and a suitable metal salt, for example manganese (II) bromide, in a suitable solvent, for example acetonitrile. Preferably the reaction is carried out at a temperature in the range −30° C. to room temperature, suitably at −20° C.

In an alternative aspect, the reaction may be carried out in the presence of a suitable chiral catalytic reagent, for example (S)-(−)-2,2′-bis(diphenylphosphino)-1,′1-binaphthyl (S-BINAP), and a suitable metal salt, for example silver acetate, in the presence of a suitable base, for example diisopropylethylamine, in a suitable solvent, for example acetonitrile optionally co-solvated with toluene. Preferably the reaction is carried out at a temperature in the range −15° C. to room temperature, suitably at −5° C.

Optionally, the major chiral diastereoisomer of a compound of Formula (IIIa) arising from such an asymmetric reaction may be further enantioenriched by conventional purification techniques well known in the art, for example by chromatography, or by fractional crystallisation. A favourable crystallisation method is the fractional crystallisation of a salt of the major chiral diastereoisomer, for example the hydrochloride salt or the (R)-(−)-1,1′-binaphthyl-2,2′-diyl-hydrogen phosphate salt.

The hydrochloride salt of a compound of Formula (IIa) may be prepared by treating a compound of Formula (IIIa) with anhydrous hydrogen chloride in a suitable solvent, for example diethyl ether. Preferably the reaction is carried out at a temperature in the range −10 to 10° C.

The (R)-(−)-1,1′-binaphthyl-2,2′-diyl-hydrogen phosphate salt of a compound of Formula (IIIa) may be prepared as herein before described for the resolution of a racemic compound of Formula (III).

It will be appreciated that, with suitable additional steps as described above, chiral compounds of Formula (Ia), (II) and/or (XI) may be prepared from chiral compounds of Formula (III), such as (IIIa), or of Formula (XII).

With appropriate manipulation and protection of any chemical functionality, synthesis of compounds of Formula (Ia) is accomplished by methods analogous to those above and to those described in the Experimental section. Suitable protecting groups can be found, but are not restricted to, those found in T W Greene and P G M Wuts ‘Protective Groups in Organic Synthesis’, 3^{rd} Ed (1999), J Wiley and Sons.

### EXAMPLES

### Intermediate 1

### 2-[N-(Diphenylmethylene)amino]-3-(1,3-thiazol-4-yl)propanoic acid, tert-butyl ester

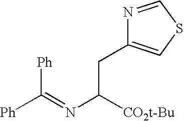

### Part A

To a cooled (ice-bath) solution of 2-[N-(diphenylmethylene)amino]ethanoic acid, tert-butyl ester (J. Org. Chem., 1982, 47, 2663; 42.3 g, 143 mmol) in dry THF (450 mL) under an atmosphere of nitrogen, was added a 1M solution of potassium t-butoxide in THF (146 mL) dropwise (dropping funnel) over 25 minutes. The mixture was allowed to stir for a further 45 minutes in the ice-bath.

### Part B

Independently during this time, 4-(chloromethyl)-1,3-thiazole hydrochloride (25.5 g, 150 mmol) was freshly converted to the free base as follows: The hydrochloride was mixed with dichloromethane (500 mL) and washed with a 5% w/v aqueous sodium bicarbonate solution (375 mL). The organic layer was separated, dried over sodium sulphate and carefully evaporated (rotary evaporator; 80 torr, water bath 25° C.) to give the free base.

### Part C

The 4-(chloromethyl)-1,3-thiazole (formed in Part B) was dissolved in THF (100 mL) and added dropwise (dropping funnel) over 30 minutes to the reaction mixture from Part A, keeping the reaction at ice-bath temperature. Solid anhydrous lithium iodide (1 g, 7.5 mmol) was added directly to the reaction mixture 5 minutes after addition of the alkylating agent had started. The dropping funnel was rinsed with further dry THF (50 mL) which was added to the reaction. The reaction was stirred at ice-bath temperature for 45 minutes, allowed to warm to room temperature over 30 minutes and was stirred at room temperature for an additional 2.5 hours before being partitioned between a mixture of saturated brine (400 mL), water (200 mL) and ethyl acetate (800 mL). The organic layer was separated and the aqueous layer re-extracted with further ethyl acetate (2×300 mL). The combined organic layers were dried over sodium sulphate and evaporated to give the title compound (57.8 g, crude) which was used without further purification.

¹H NMR (CDCl₃): δ 8.65 (d, 1H), 7.55-7.62 (m, 2H), 7.2-7.55 (m, 6H), 7.05 (d, 1H), 6.78-6.87 (m, 2H), 4.36-4.41 (m, 1H), 3.47-3.54 (m, 1H), 3.36-3.44 (m, 1H) and 1.44 (s, 9H).

### Intermediate 2

### 2-Amino-3-(1,3-thiazol-4-yl)propanoic acid, tert-butyl ester

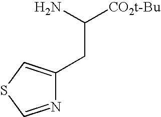

To a solution of 2-[N-(diphenylmethylene)amino]-3-(1,3-thiazol-4-yl)propanoic acid, tert-butyl ester (prepared in a similar manner to that described in Intermediate 1; 20 g) in THF (150 mL) under argon was added a solution of citric acid in water (15% w/v, 150 mL). The mixture was stirred at room temperature for 6 hours, left overnight and then the majority of the THF was removed under reduced pressure (rotary evaporator; water bath at 25° C.) and 1 M aqueous hydrochloric acid (60 mL) added. The mixture was extracted with diethyl ether (2×200 mL) and the combined ether extracts back extracted with water (50 mL). The combined aqueous layers were extracted with further diethyl ether (100 mL). All of the ether layers were discarded. The aqueous layer was then carefully adjusted to pH 9.5 with potassium carbonate, brine (100 mL) was added and the mixture extracted with diethyl ether (4×200 mL). These combined ether layers were dried over sodium sulphate. Removal of the solvent under reduced pressure gave the title compound, an oil.

¹H NMR (CDCl₃): δ 8.77 (d, 1H), 7.08 (d, 1H), 3.77-3.85 (m, 1H), 3.22-3.32 (m, 1H), 3.02-3.13 (m, 1H) and 1.42 (s, 9H). Amine protons not observed.

### Intermediate 3

### 2-[N-(1,3-Thiazol-2-ylmethylene)amino]-3-(1,3-thiazol-4-yl)propanoic acid, tert-butyl ester

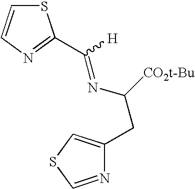

A mixture of 2-amino-3-(1,3-thiazol-4-yl)propanoic acid, tert-butyl ester (Intermediate 2, 9.13 g, 40 mmol), 1,3-thiazole-2-carboxaldehyde (4.52 g, 40 mmol) and magnesium sulfate (ca. 5 g) in dichloromethane (80 mL) was stirred at room temperature for 18 hours. The reaction mixture was filtered, and the filtrate was evaporated to give the title compound as an oil.

¹H NMR (CDCl₃): δ 8.75 (d, 1H), 8.23 (s, 1H), 7.90 (d, 1H), 7.43 (dd, 1H), 7.03 (d, 1H), 4.50 (dd, 1H), 3.58 (dd, 1H), 3.34 (dd, 1H) and 1.45 (s, 9H).

### Intermediate 4

### rel-(2R,4S,5R)-4-(Pyrazin-2-yl)-2-(1,3-thiazol-4-ylmethyl)-5-(1,3-thiazol-2-yl)-pyrrolidine-2-carboxylic acid, tert-butyl ester

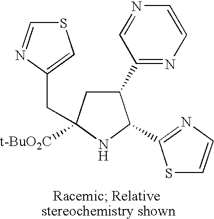

A mixture of 2-[N-(1,3-thiazol-2-ylmethylene)amino]-3-(1,3-thiazol-4-yl)propanoic acid, tert-butyl ester (Intermediate 3, 13.72 g, 40 mmol), vinyl pyrazine (4.24 g, 40 mmol), lithium bromide (6.96 g, 80 mmol), and triethylamine (8.40 mL, 60 mmol) in tetrahydrofuran (110 mL) was stirred at room temperature under nitrogen for 24 hours. A saturated solution of ammonium chloride (250 mL) was added and the mixture was extracted with ethyl acetate (2×300 mL). Extracts were washed with water and brine, dried over magnesium sulfate and evaporated. The residue was purified by chromatography on silica gel using ethyl acetate-methanol (95:5 v/v) as eluent to give the title compound as a gum.

MS calcd for (C₂₀H₂₃N₅O₂S₂+H)⁺: 430

MS found (electrospray): (M+H)⁺=430

### Intermediate 5

### rel-(2R,4S,5R)-1-(3-methoxy-4-tert-butylbenzoyl)-4-(pyrazin-2-yl)-2-(1,3-thiazol-4-ylmethyl)-5-(1,3-thiazol-2-yl)pyrrolidine-2-carboxylic acid, tert-butyl ester

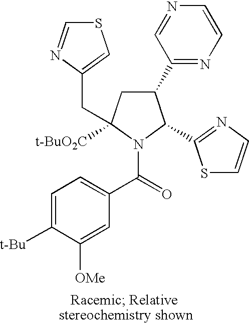

A mixture of rel-(2R,4S,5R)-4-(pyrazin-2-yl)-2-(1,3-thiazol-4-ylmethyl)-5-(1,3-thiazol-2-yl)pyrrolidine-2-carboxylic acid, tert-butyl ester (Intermediate 4, 10.90 g, 25.4 mmol), 3-methoxy-4-tert-butylbenzoyl chloride (9.10 g, 43.2 mmol) and triethylamine (9.71 mL) in dichloromethane (200 mL) was stirred at room temperature for 4 hours. The mixture was washed with water, dried by passing through a hydrophobic frit and evaporated. The residue was purified by chromatography on silica gel using cyclohexane-ethyl acetate (2:1 v/v) followed by ethyl acetate as eluent to give the title compound as a solid.

MS calcd for (C₃₂H₃₇N₅O₄S₂+H)⁺: 620

MS found (electrospray): (M+H)⁺=620

### Intermediate 6

### 2-[N-(Diphenylmethylene)amino]-3-(1,3-thiazol-2-yl)propanoic acid, tert-butyl ester

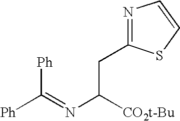

The title compound was prepared in a similar manner to Intermediate 1, using 2-(chloromethyl)-1,3-thiazole in place of 4-(chloromethyl)-1,3-thiazole.

¹H NMR (CDCl₃): δ 7.65 (m, 3H), 7.40-7.30 (m, 6H), 7.19 (d, 1H), 6.91 (m, 2H), 4.38 (dd, 1H), 3.65 (m, 2H) and 1.43 (s, 9H).

### Intermediate 7

### 2-Amino-3-(1,3-thiazol-2-yl)propanoic acid, tert-butyl ester

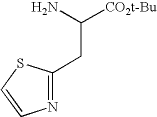

The title compound was prepared in a similar manner to Intermediate 2, using Intermediate 6 in place of Intermediate 1.

¹H NMR (CDCl₃): δ 7.70 (d, 1H), 7.21 (d, 1H), 3.79 (dd, 1H), 3.42 (dd, 1H), 3.24 (dd, 1H), 1.74 (broad s, 2H) and 1.42 (s, 9H).

### Intermediate 8

### 2-[N-(1,3-Thiazol-2-ylmethylene)amino]-3-(1,3-thiazol-2-yl)propanoic acid, tert-butyl ester

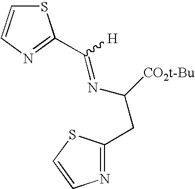

The title compound was prepared in a similar manner to Intermediate 3, using Intermediate 7 in place of Intermediate 2.

¹H NMR (CDCl₃): δ 8.39 (s, 1H), 7.93 (d, 1H), 7.70 (d, 1H), 7.46 (d, 1H), 7.19 (d, 1H), 4.51 (dd, 1H), 3.77 (dd, 1H), 3.59 (dd, 1H) and 1.45 (s, 9H).

### Intermediate 9

### rel-(2R,4S,5R)-4-(Pyrazin-2-yl)-2-(1,3-thiazol-2-ylmethyl)-5-(1,3-thiazol-2-yl)-pyrrolidine-2-carboxylic acid, tert-butyl ester

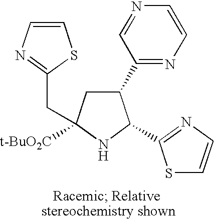

The title compound was prepared in a similar manner to Intermediate 4, using Intermediate 8 in place of Intermediate 3.

MS calcd for (C₂₀H₂₃N₅O₂S₂+H)⁺: 430

MS found (electrospray): (M+H)⁺=430.

### Intermediate 10

### rel-(2R,4S,5R)-1 (3-Methoxy-4-tert-butylbenzoyl)-4-(pyrazin-2-yl)-2-(1,3-thiazol-2-ylmethyl)-5-(1,3-thiazol-2-yl)pyrrolidine-2-carboxylic acid, tert-butyl ester

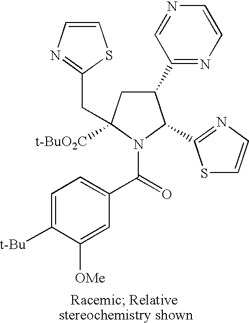

The title compound was prepared in a similar manner to Intermediate 5, using Intermediate 9 in place of Intermediate 4.

MS calcd for (C₃₂H₃₇N₅O₄S₂+H)⁺: 620

MS found (electrospray): (M+H)⁺=620.

### Intermediate 11

### 2-[N-(Diphenylmethylene)amino]-3-(1,2-thiazol-3-yl)propanoic acid, tert-butyl ester

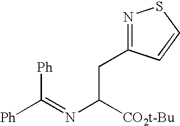

The title compound was prepared in a similar manner to Intermediate 1, using 3-(bromomethyl)-1,2-thiazole in place of 4-(chloromethyl)-1,3-thiazole.

¹H NMR (CDCl₃): δ 8.51 (d, 1H), 7.61-7.18 (m, 8H), 7.08 (dd, 1H), 6.83 (m, 2H), 4.38 (dd, 1H), 3.48 (d, 2H) and 1.43 (s, 9H).

### Intermediate 12

### 2-Amino-3-(1,2-thiazol-3-yl)propanoic acid, tert-butyl ester

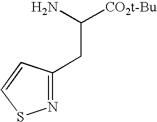

The title compound was prepared in a similar manner to Intermediate 2, using Intermediate 11 in place of Intermediate 1.

¹H NMR (CDCl₃): δ 8.60 (d, 1H), 7.13 (d, 1H), 3.84 (dd, 1H), 3.29 (dd, 1H), 3.15 (dd, 1H) and 1.43 (s, 9H).

### Intermediate 13

### 2-[[N-(5-Methyl-1,3-thiazol-2-yl)methylene]amino]-3-(1,2-thiazol-3-yl)propanoic acid, tert-butyl ester

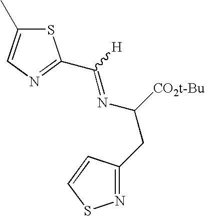

The title compound was prepared in a similar manner to Intermediate 3, using Intermediate 12 in place of Intermediate 2, and 5-methyl-1,3-thiazole-2-carboxaldehyde in place of 1,3-thiazole-2-carboxaldehyde.

¹H NMR (CDCl₃): δ 8.53 (d, 1H), 8.23 (d, 1H), 7.56 (d, 1H), 7.08 (d, 1H), 4.50 (dd, 1H), 3.57 (dd, 1H), 3.39 (dd, 1H), 2.50 (d, 3H) and 1.44 (s, 9H).

### Intermediate 14

### rel-2R,4S,5R)-5-(5-Methyl-1,3-thiazol-2-yl)-4-(pyrazin-2-yl)-2-(1,2-thiazol-3-yl-methyl)-pyrrolidine-2-carboxylic acid, tert-butyl ester

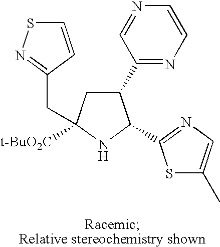

The title compound was prepared in a similar manner to Intermediate 4, using Intermediate 13 in place of Intermediate 3.

MS calcd for (C₂₁H₂₅N₅O₂S₂+H)⁺: 444

MS found (electrospray): (M+H)⁺=444

### Intermediate 15

### rel-(2R,4S,5R)-1-(3-Methoxy-4-tert-butylbenzoyl)-5-(5-methyl-1,3-thiazol-2-yl)-4-(pyrazin-2-yl)-2-(1,2-thiazol-3-ylmethyl)pyrrolidine-2-carboxylic acid, tert-butyl ester

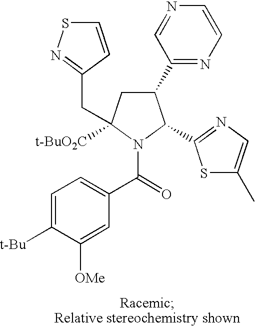

The title compound was prepared in a similar manner to Intermediate 5, using Intermediate 14 in place of Intermediate 4.

MS calcd for (C₃₃H₃₉N₅O₄S₂+H)⁺: 634

MS found (electrospray): (M+H)⁺=634

### Intermediate 16

### 2-[N-(1,3-Thiazol-2-ylmethylene)amino]-3-(1,2-thiazol-3-yl)propanoic acid, tert-butyl ester

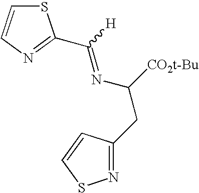

The title compound was prepared in a similar manner to Intermediate 3, using Intermediate 12 in place of Intermediate 2.

¹H NMR (CDCl₃): δ 8.54 (d, 1H), 8.34 (s, 1H), 7.92 (d, 1H), 7.44 (dd, 1H), 7.10 (d, 1H), 4.54 (dd, 1H), 3.60 (dd, 1H), 3.42 (dd, 1H) and 1.43 (s, 9H).

### Intermediate 17

### rel-(2R,4S,5R)-4-(Pyrazin-2-yl)-2-(1,2-thiazol-3-ylmethyl)-5-(1,3-thiazol-2-yl)-pyrrolidine-2-carboxylic acid, tert-butyl ester

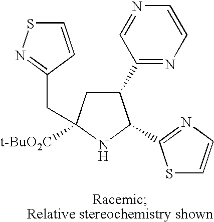

The title compound was prepared in a similar manner to Intermediate 4, using Intermediate 16 in place of Intermediate 3.

MS calcd for (C₂₀H₂₃N₅O₂S₂+H)⁺: 430

MS found (electrospray): (M+H)⁺=430

### Intermediate 18

### rel-(2R,4S,5R)-1-(3-Methoxy-4-tert-butylbenzoyl)-4-(pyrazin-2-yl)-2-(1,2-thiazol-3-ylmethyl)-5-(1,3-thiazol-2-yl)pyrrolidine-2-carboxylic acid, tert-butyl ester

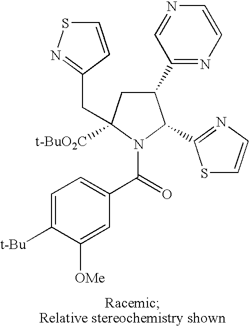

The title compound was prepared in a similar manner to Intermediate 5, using Intermediate 17 in place of Intermediate 4.

MS calcd for (C₃₂H₃₇N₅O₄S₂+H)⁺: 620

MS found (electrospray): (M+H)⁺=620

### Intermediate 19

### 2-Amino-3-(1H-pyrazol-1-yl)propanoic acid, tert-butyl ester

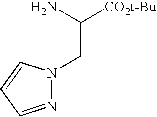

To a stirred suspension of 2-amino-3-(1H-pyrazol-1-yl)propanoic acid (10.2 g, 65.9 mmol) in tert-butyl acetate (400 mL) was added a solution of 70% aqueous perchloric acid (15.7 mL). The mixture was allowed to stir at room temperature for 0.5 hours and was then allowed to stand for 20 hours. The reaction mixture was diluted with ethyl acetate and then neutralised using a combination of saturated aqueous sodium bicarbonate and solid sodium bicarbonate. The aqueous phase was separated off and extracted with ethyl acetate. The organic phases were combined, dried over MgSO₄ and evaporated to give the title compound, an oil.

MS calcd for (C₁₀H₁₇N₃O₂+H)⁺: 212

MS found (electrospray): (M+H)⁺=212

### Intermediate 20

### 2-[[N-(5-Methyl-1,3-thiazol-2-yl)methylene]amino]-3-(1H-pyrazol-1-yl)propanoic acid, tert-butyl ester

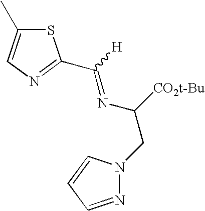

The title compound was prepared in a similar manner to Intermediate 3, using Intermediate 19 in place of Intermediate 2 and 5-methyl-1,3-thiazole-2-carboxaldehyde in place of 1,3-thiazole-2-carboxaldehyde.

¹H NMR (CDCl₃): δ 7.98 (s, 1H), 7.56 (bd, 1H), 7.50 (bd, 1H), 7.34 (d, 1H), 6.13 (t, 1H), 4.82-4.73 (m, 1H), 4.50-4.43 (m, 2H), 2.51 (s, 3H) and 1.47 (s, 9H).

### Intermediate 21

### rel-(2R,4S,5R)-5-(5-Methyl-1,3-thiazol-2-yl)-4-(pyrazin-2-yl)-2-(1H-pyrazol-1-yl-methyl)pyrrolidine-2-carboxylic acid, tert-butyl ester

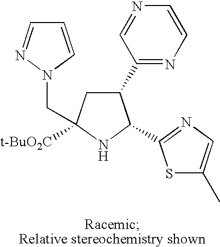

The title compound was prepared in a similar manner to Intermediate 4, using Intermediate 20 in place of Intermediate 3.

MS calcd for (C₂₁H₂₁N₂₆N₆O₂S+H)⁺: 427

MS found (electrospray): (M+H)⁺=427

### Intermediate 22

### rel-(2R,4S,5R)-1-(3-Methoxy-4-tert-butylbenzoyl)-5-(5-methyl-1,3-thiazol-2-yl)-4-(pyrazin-2-yl)-2-(1H-pyrazol-1-ylmethyl)pyrrolidine-2-carboxylic acid, tert-butyl ester

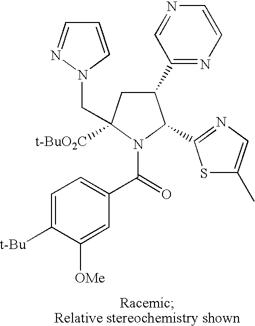

The title compound was prepared in a similar manner to Intermediate 5, using Intermediate 21 in place of Intermediate 4.

MS calcd for (C₃₃H₄₀N₆O₄S+H)⁺: 617

MS found (electrospray): (M+H)⁺=617

### Intermediate 23

rel-(2R,4S,5R)-4-(Pyrazin-2-yl)-2-(1H-pyrazol-1-ylmethyl)-5-(1,3-thiazol-2-yl)-pyrrolidine-2-carboxylic acid, tert-butyl ester

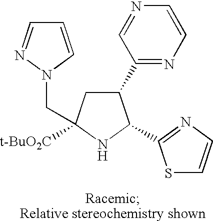

Molecular sieves (3A, 0.75 g) were added to a solution of 1,3-thiazole-2-carboxaldehyde (0.170 mL) and Intermediate 19 (317 mg) in dry tetrahydrofuran (4.5 mL). The mixture was stirred at room temperature, under nitrogen for 18 hours and then treated with lithium-bromide (260 mg), vinyl pyrazine (191 mg) and triethylamine (0.251 mL). The mixture was stirred overnight at room temperature, under nitrogen. Ethyl acetate (10 mL) was added and the molecular sieves were removed by filtration. The filtrate was washed with saturated ammonium chloride solution (10 mL), the organic solution was separated and the aqueous solution extracted with ethyl acetate (20 mL). The combined organic extracts were dried over magnesium sulfate, filtered and evaporated under reduced pressure. The residue was purified by chromatography on silica gel using ethyl acetate as eluent to give the title compound as a solid.

MS calcd for (C₂₀H₂₄N₆O₂S+H)⁺: 413

MS found (electrospray): (M+H)⁺=413

### Intermediate 24

### rel-(2R,4S,5R)-1-(3-Methoxy-4-tert-butylbenzoyl)-4-(pyrazin-2-yl)-2-(1H-pyrazol-1-ylmethyl)-5-(1,3-thiazol-2-yl)pyrrolidine-2-carboxylic acid, tert-butyl ester

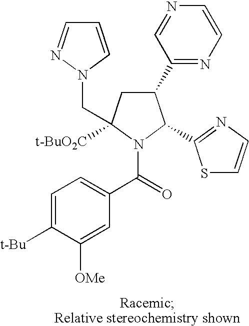

The title compound was prepared in a similar manner to Intermediate 5, using Intermediate 23 in place of Intermediate 4.

MS calcd for (C₃₂H₃₈N₆O₄S+H)⁺: 603

MS found (electrospray): (M+H)⁺=603

### Intermediate 25

### 2-[[N-(5-Methyl-1,3-thiazol-2-yl)methylene]amino]-3-(1,3-thiazol-2-yl)propanoic acid, tert-butyl ester

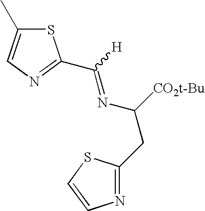

The title compound was prepared in a similar manner to Intermediate 3, using Intermediate 7 in place of Intermediate 2, and 5-methyl-1,3-thiazole-2-carboxaldehyde in place of 1,3-thiazole-2-carboxaldehyde.

¹H NMR (CDCl₃): δ 8.28 (s, 1H), 7.69 (d, 1H), 7.60 (d, 1H), 7.19 (d, 1H), 4.46 (m, 1H), 3.75 (dd, 1H), 3.58 (dd, 1H), 2.50 (s, 3H) and 1.42 (s 9H).

### Intermediate 26

### rel-(2R,4S,5R)-5-(5-Methyl-1,3-thiazol-2-yl)-4-(pyrazin-2-yl)-2-(1,3-thiazol-2-yl-methyl)pyrrolidine-2-carboxylic acid, tert-butyl ester

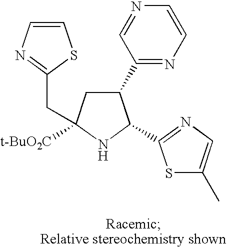

The title compound was prepared in a similar manner to Intermediate 4, using Intermediate 25 in place of Intermediate 3.

MS calcd for (C₂₁H₂₅N₅O₂S₂+H)⁺: 444

MS found (electrospray): (M+H)⁺=444

### Intermediate 27

### rel-(2R,4S,5R)-1-(3-Methoxy-4-tert-butylbenzoyl)-5-(5-methyl-1,3-thiazol-2-yl)-4-(pyrazin-2-yl)-2-(1,3-thiazol-2-ylmethyl)pyrrolidine-2-carboxylic acid, tert-butyl ester

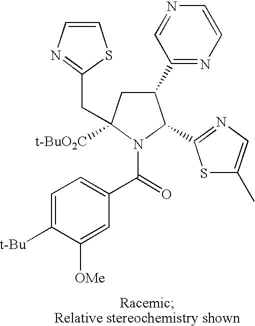

The title compound was prepared in a similar manner to Intermediate 5, using Intermediate 26 in place of Intermediate 4.

MS calcd for (C₃₃H₃₉N₅O₄S₂+H)⁺: 634

MS found (electrospray): (M+H)⁺=634

### Intermediate 28

### 2-[[N-(5-Methyl-1,3-thiazol-2-yl)methylene]amino]-3-(1,3-thiazol-4-yl)propanoic acid, tert-butyl ester

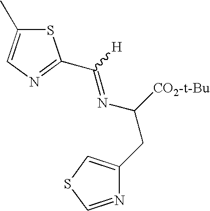

The title compound was prepared in a similar manner to Intermediate 3, using 5-methyl-1,3-thiazole-2-carboxaldehyde in place of 1,3-thiazole-2-carboxaldehyde.

¹H NMR (CDCl₃): δ 8.77 (d, 1H), 8.11 (s, 1H), 7.55 (br s, 1H), 7.03 (d, 1H), 4.45 (dd, 1H), 3.56 (dd, 1H), 3.32 (m, 1H), 2.50 (s, 3H) and 1.44 (s, 9H).

### Intermediate 29

### rel-(2R,4S,5R)-5-(5-Methyl-1,3-thiazol-2-yl)-4-(pyrazin-2-yl)-2-(1,3-thiazol-4-yl-methyl)pyrrolidine-2-carboxylic acid, tert-butyl ester

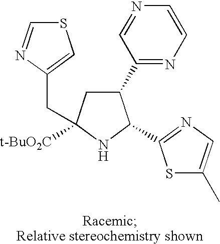

The title compound was prepared in a similar manner to Intermediate 4, using Intermediate 28 in place of Intermediate 3.

MS calcd for (C₂₁H₂₅N₅O₂S₂+H)⁺: 444

MS found (electrospray): (M+H)⁺=444.

### Intermediate 30

### rel-(2R,4S,5R)-1-(3-Methoxy-4-tert-butylbenzoyl)-5-(5-methyl-1,3-thiazol-2-yl)-4-(pyrazin-2-yl)-2-(1,3-thiazol-4-ylmethyl)pyrrolidine-2-carboxylic acid, tert-butyl ester

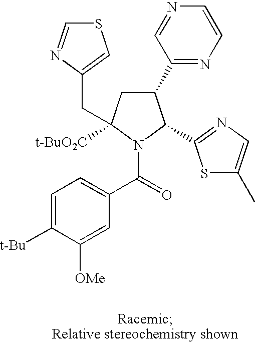

The title compound was prepared in a similar manner to Intermediate 5, using Intermediate 29 in place of Intermediate 4.

MS calcd for (C₃₃H₃₉N₅O₄S₂+H)⁺: 634

MS found (electrospray): (M+H)⁺=634

### Intermediate 31

### Chiral Diastereoisomeric Salt A of rel-(2R,4S,5R)-4-(Pyrazin-2-yl)-2-(1,3-thiazol-4-ylmethyl)-5-(1,3-thiazol-2-yl)pyrrolidine-2-carboxylic acid, tert-butyl ester with (R)-(−)-1,1′-binaphthyl-2,2′-diyl-hydrogen phosphate

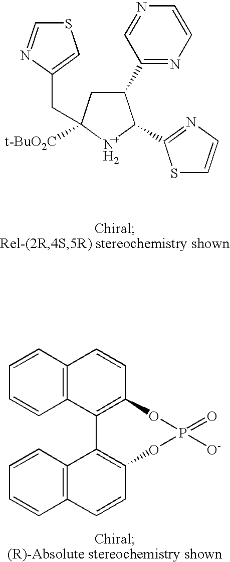

rel-(2R,4S,5R)-4-(Pyrazin-2-yl)-2-(1,3-thiazol-4-ylmethyl)-5-(1,3-thiazol-2-yl)-pyrrolidine-2-carboxylic acid, tert-butyl ester (Intermediate 4, 10.8 g, 25.1 mmol) and (R)-1,1′-binaphthyl-2,2′-diyl-hydrogen phosphate (8.76 g, 25.1 mmol) were suspended in acetonitrile (55 mL) and heated to reflux with stirring to give a clear solution. After being allowed to cool to room temperature, the mixture was stirred with ice cooling for a further 1 hour and the precipitated solid filtered then slurried with a further portion of acetonitrile (55 mL). The mixture was stirred with ice cooling for a further 1 hour, the solid was filtered, washed with ice cold acetonitrile (55 mL) and dried at 40° C. under vacuum to afford the title compound.

¹H NMR (CDCl₃): δ 8.75 (d, 1H), 8.30 (m, 2H), 8.23 (s, 1H), 7.88 (m, 4H), 7.54 (m, 2H), 7.42 (m, 5H), 7.35 (d, 1H), 7.25 (m, 2H), 6.88 (s, 1H), 5.64 (m, 1H), 4.25 (m, 1H), 3.68 (m, 2H), 3.17 (m, 1H), 2.90 (m, 1H) and 1.41 (s, 9H). Amine protons not seen.

### Intermediate 32

### Enantiomer A of rel-(2R,4S,5R)-4-(Pyrazin-2-yl)-2-(1,3-thiazol-4-ylmethyl)-5-(1,3-thiazol-2-yl)pyrrolidine-2-carboxylic acid, tert-butyl ester

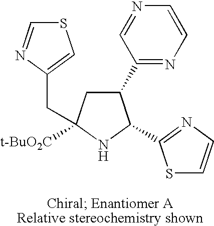

Intermediate 31 (15 g, 19.3 mmol) was suspended in methyl tert-butyl ether (150 mL) and triethylamine (2.96 mL, 21.2 mmol) added with stirring. After 1 hour the solid was filtered off and washed with methyl tert-butyl ether (150 mL). The combined filtrate was evaporated to an oil, which crystallised on trituration with cyclohexane. The solid was filtered, washed and dried under vacuum to give the title compound.

¹H NMR (CDCl₃); δ 8.76 (d, 1H), 8.30 (dd, 1H), 8.24 (d, 1H), 8.16 (d, 1H), 7.46 (d, 1H), 7.23 (d, 1H), 7.02 (d, 1H), 5.03 (d, 1H), 3.91 (dd, 1H), 3.66 (br, 1H), 3.41 (d, 1H), 3.35 (d, 1H), 3.07 (dd, 1H), 2.55 (dd, 1H) and 1.46 (s, 9H).

¹H NMR showed that this compound was identical to the corresponding racemate, described in Intermediate 4. Comparison of Intermediate 32 with the corresponding racemate Intermediate 4 by analytical chiral HPLC on a Chiralcell OD-H column, us ing heptane-ethanol (1:1 v/v) as eluent showed the title compound to be the first eluting enantiomer.

### Intermediate 33

### Enantiomer A of rel-(2R,4S,5R)-1-(3-methoxy-4-tert-butylbenzoyl)-4-(pyrazin-2-yl)-2-(1,3-thiazol-4-ylmethyl)-5-(1,3-thiazol-2-yl)pyrrolidine-2-carboxylic acid, tert-butyl ester

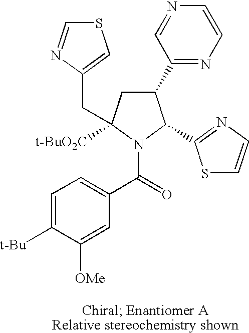

The title compound was prepared in a similar manner to Intermediate 5, using Intermediate 32 in place of Intermediate 4.

MS calcd for (C₃₂H₃₇N₅O₄S₂+H)⁺: 620

MS found (electrospray): (M+H)⁺=620

¹H NMR showed that this compound was identical to the corresponding racemate, described in Intermediate 5.

### Intermediate 34

### Enantiomer A of rel-(2R,4S,5R)-4-(Pyrazin-2-yl)-2-(1,3-thiazol-4-ylmethyl)-5-(1,3-thiazol-2-yl)pyrrolidine-2-carboxylic acid, tert-butyl ester

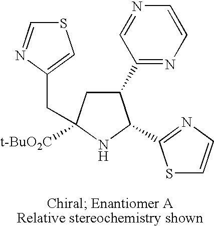

To a stirred solution of Intermediate 3 (11.8 g) in acetonitrile (177 mL) was added portion wise manganese (II) bromide (15.7 g). The addition was accompanied by a mild exotherm. The reaction mixture was cooled to a temperature of 20° C. in an ice-water bath and (1R,2S)-(−)N-methylephedrine (9.8 g) was added. The mixture was stirred for 5 minutes. Vinyl pyrazine (5.8 g) was then added and the mixture was stirred at room temperature overnight before being diluted with ethyl acetate and poured into an aqueous solution of ammonium chloride. The organic layer was separated and the aqueous layer was re-extracted with ethyl acetate. The organic layers were combined and dried over sodium sulphate. The sodium sulphate was removed by filtration and the filtrate was then evaporated under reduced pressure to give the crude product (18.9 g) as an oil. The crude product was purified by column chromatography on silica gel, eluting initially with dichloromethane, followed by ethyl acetate, and finally with ethyl acetate-methanol (9:1 v/v) to give the title compound as a gum.

MS calcd for (C₂₀H₂₃N₅S₂O₂+H)⁺: 430

MS found (electrospray): (M+H)⁺=430

¹H NMR showed that this compound was identical to that prepared by salt resolution, described in Intermediate 32, and to the racemate described in Intermediate 4. Comparison of Intermediate 34 with the corresponding racemate Intermediate 4 by analytical chiral HPLC on a Chiralcell OD-H column, using heptane-ethanol (1:1 v/v) as eluent showed the title compound to be the first eluting enantiomer.

### Example 1

### rel-(2R,4S,5R)-1-(3-Methoxy-4-tert-butylbenzoyl)-4-(pyrazin-2-yl)-2-(1,3-thiazol-4-ylmethyl)-5-(1,3-thiazol-2-yl)pyrrolidine-2-carboxylic acid

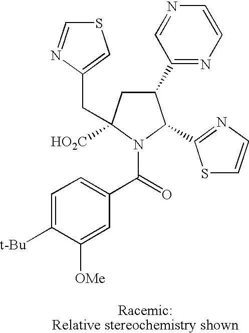

A solution of rel-(2R,4S,5R)-1-(3-methoxy-4-tert-butylbenzoyl)-4-(pyrazin-2-yl)-2-(1,3-thiazol-4-ylmethyl)-5-(1,3-thiazol-2-yl)pyrrolidine-2-carboxylic acid, tert-butyl ester (Intermediate 5, 13.909, 22.4 mmol) in dichloromethane (100 mL) was treated with trifluoroacetic acid (85 mL) at room temperature for 4 hours. The mixture was evaporated to dryness. The crude product was dissolved in dichloromethane (250 mL) and washed with water until the washings were at pH 7. The organic phase was dried (hydrophobic frit) and evaporated. The residue was triturated with ether to give the title compound as a solid.

MS calcd for (C₂₈H₂₉N₅O₄S₂+H)⁺: 564

MS found (electrospray): (M+H)⁺=564.

¹H NMR (CDCl₃): δ 14.61 (s, 1H), 8.93 (d, 1H), 8.31 (d, 1H), 8.25 (dd, 1H), 8.08 (d, 1H), 7.73 (d, 1H), 7.36 (d, 1H), 7.13 (d, 1H), 7.04 (d, 1H), 6.67 (dd, 1H), 6.43 (d, 1H), 5.39 (d, 1H), 4.35 (d, 1H), 3.82 (d, 1H), 3.60 (s, 3H), 3.35 (m, 1H), 3.21 (t, 1H), 2.78 (dd, 1H), 1.27 (s, 9H).

### Example 2

### Enantiomer A of rel-(2R,4S,5R)-1-(3-Methoxy-4-tert-butylbenzoyl)-4-pyrazin-2-yl)-2-(1,3-thiazol-4-ylmethyl)-5-(1,3-thiazol-2-yl)pyrrolidine-2-carboxylic acid

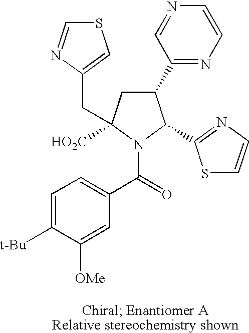

rel-(2R,4S,5R)-1-(3-Methoxy-4-tert-butylbenzoyl)-4-(pyrazin-2-yl)-2-(1,3-thiazol-4-yl-methyl)-5-(1,3-thiazol-2-yl)pyrrolidine-2-carboxylic acid (Example 1) was resolved by preparative chiral HPLC on a Chiralpak AD column using heptane-ethanol (70:30 v/v) containing 0.1% trifluoroacetic acid as eluent. Column fractions containing the second eluting enantiomer were combined and evaporated. The second eluting enantiomer was dissolved in dichloromethane, washed with water; dried (hydrophobic frit) and solvent removed to give the title compound.

MS calcd for (C₂₈H₂₉N₅O₄S₂+H)⁺: 564

MS found (electrospray): (M+H)⁺=564.

¹H NMR showed that this compound was identical to the compound of Example 1.

### Example 3

### rel-(2R,4S,5R)-1-(3-Methoxy-4-tert-butylbenzoyl)-4-(pyrazin-2-yl)-2-(1,3-thiazol-2-ylmethyl)-5-(1,3-thiazol-2-yl)pyrrolidine-2-carboxylic acid

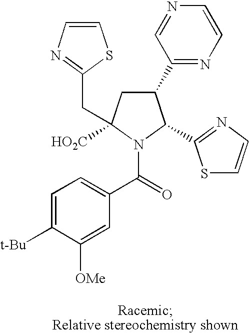

The crude product was obtained in a similar manner to Example 1, using Intermediate 10 in place of Intermediate 5, and was triturated with ether to give the title compound.

MS calcd for (C₂₈H₂₉N₅O₄S₂+H)⁺: 564

MS found (electrospray): (M+H)⁺=564

¹H NMR (CD₃OD): δ 8.29 (m, 2H), 8.22 (m, 1H), 7.96 (d, 1H), 7.74 (d, 1H), 7.68 (d, 1H), 7.34 (d, 1H), 7.18 (d, 1H), 6.82 (d, 1H), 6.59 (s, 1H), 5.77 (d, 1H), 4.32 (d, 1H), 3.96 (d, 1H), 3.67 (s, 3H), 3.46 (m, 1H), 3.32 (m, 1H), 2.67 (dd, 1H), 1.27 (s, 9H). Carboxylic acid proton exchanged with solvent.

### Example 4

### Enantiomer A of rel-(2R,4S,5R)-1-(3-methoxy-4-tert-butylbenzoyl)-4-(pyrazin-2-yl)-2-(1,3-thiazol-2-ylmethyl)-5-(1,3-thiazol-2-yl)pyrrolidine-2-carboxylic acid

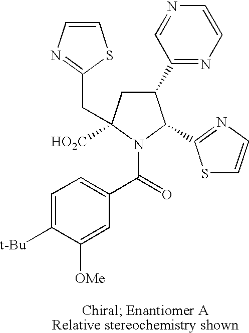

rel-(2R,4S,5R)-1-(3-Methoxy-4-tert-butylbenzoyl)-4-(pyrazin-2-yl)-2-(1,3-thiazol-2-yl-methyl)-5-(1,3-thiazol-2-yl)pyrrolidine-2-carboxylic acid (Example 3) was resolved by preparative chiral HPLC on a Chiralpak AD column using heptane-ethanol (70:30 v/v) containing 0.1% trifluoroacetic acid as eluent to afford the first and second eluting enantiomers. The fractions containing the second eluting enantiomer were combined and evaporated. This crude product was dissolved in dichloromethane, washed with sodium hydrogen carbonate solution; dried (hydrophobic frit) and solvent removed to give the title compound

MS calcd for (C₂₈H₂₉N₅O₄S₂+H)⁺: 564

MS found (electrospray): (M+H)⁺=564

¹H NMR (CD₃OD): δ 8.12 (s, 2H), 8.01 (s, 1H), 7.89 (d, 1H), 7.66 (d, 1H), 7.29 (d, 1H), 7.14 (d, 1H), 6.95 (m, 2H), 6.75 (s, 1H), 5.44 (d, 1H), 4.48 (d, 1H), 4.02 (d, 1H), 3.57 (s, 3H), 3.36 (t, 1H), 3.03 (m, 1H), 2.55 (dd, 1H) and 1.25 (s, 9H). Carboxylic acid proton exchanged with solvent.

### Example 5

### rel-(2R,4S,5R)-1-(3-Methoxy-4-tert-butylbenzoyl)-5-(5-methyl-1,3-thiazol-2-yl)-4-(pyrazin-2-yl)-2-(1,2-thiazol-3-ylmethyl)pyrrolidine-2-carboxylic acid

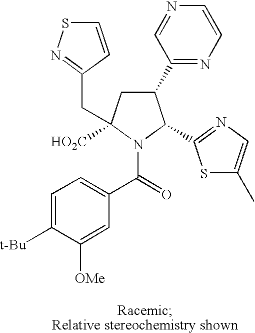

The crude product was obtained in a similar manner to Example 1, using Intermediate 15 in place of Intermediate 5, and was dissolved in dichloromethane and washed with sodium hydrogen carbonate solution, dried (hydrophobic frit) and the solvent removed to give the title compound.

MS calcd for (C₂₉H₃₁N₅O₄S₂+H)⁺: 578

MS found (electrospray): (M+H)⁺=578

¹H NMR (CDCl₃): δ8.74 (1H, d), 8.32 (1H, d), 8.28 (1H, t), 8.08 (1H, d), 7.37 (1H, d), 7.35 (1H, bd), 7.15 (1H, d), 6.70 (1H, dd), 6.45 (1H, d), 5.29 (1H, d), 4.33 (1H, d), 3.63 (3H, s), 3.85 (1H, d), 3.23-3.09 (2H, m), 2.76-2.69 (1H, m), 2.18 (3H, s) and 1.28 (9H, s). Carboxylic acid proton not seen.

### Example 6

### Enantiomer A of rel-(2R,4S,5R)-1-(3-Methoxy-4-tert-butylbenzoyl)-5-(5-methyl-1,3-thiazol-2-yl)-4-(pyrazin-2-yl)-2-(1,2-thiazol-3-ylmethyl)pyrrolidine-2-carboxylic acid

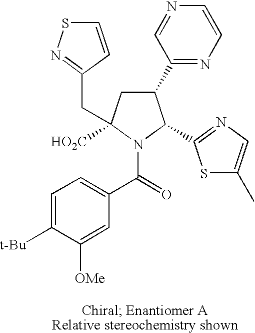

rel-(2R,4S,5R)-1-(3-Methoxy-4-tert-butylbenzoyl)-5-(5-Methyl-1,3-thiazol-2-yl)-4-(pyrazin-2-yl)-2-(1,2-thiazol-3-ylmethyl)pyrrolidine-2-carboxylic acid (Example 5) was resolved by preparative chiral HPLC on a Chiralpak AD column using heptane-ethanol (60:40 v/v) containing 0.1% trifluoroacetic acid as eluent. The fractions containing the second eluting enantiomer were combined and evaporated. This crude product was dissolved in dichloromethane, washed with sodium hydrogen carbonate solution; dried (hydrophobic frit) and solvent removed to give the title compound.

MS calcd for (C₂₉H₃₁N₅O₄S₂+H)⁺: 578

MS found (electrospray): (M+H)⁺=578.

¹H NMR (CDCl₃): δ 8.74 (d, 1H), 8.32 (d, 1H), 8.28 (t, 1H), 8.08 (d, 1H), 7.38 (d, 1H), 7.35 (d, 1H), 7.15 (d, 1H), 6.70 (dd, 1H), 6.45 (d, 1H), 5.29 (d, 1H), 4.33 (d, 1H), 3.84 (d, 1H), 3.63 (s, 3H), 3.22-3.10 (m, 2H), 2.78-2.68 (m, 1H), 2.18 (d, 3H) and 1.28 (s, 9H). Acid proton not seen.

### Example 7

### rel-(2R,4S,5R)-1-(3-Methoxy-4-tert-butylbenzoyl)-4-(pyrazin-2-yl)-2-(1,2-thiazol-3-ylmethyl)-5-(1,3-thiazol-2-yl)pyrrolidine-2-carboxylic acid

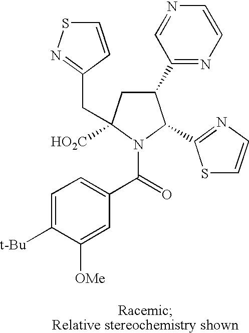

The crude product was obtained in a similar manner to Example 1, using Intermediate 18 in place of Intermediate 5. The crude product was purified by column chromatography on silica gel, eluting initially with cyclohexane-ethyl acetate (gradient elution from 50:1 v/v to 1:9 v/v) followed by dichloromethane-methanol (9:1 v/v) to give the title compound.

MS calcd for (C₂₈H₁₉N₅O₄S₂+H)⁺: 564

MS found (electrospray): (M+H)⁺=564

¹H NMR (CD₃OD): δ 9.00 (d, 1H), 8.25 (d, 1H), 8.19 (d, 2H), 7.64 (d, 1H), 7.49 (d, 1H), 7.31 (d, 1H), 7.15 (d, 1H), 6.71 (dd, 1H), 6.66 (d, 1H), 5.60 (d, 1H), 4.16 (d, 1H), 3.73 (d, 1H), 3.70 (s, 3H), 3.25 (m, 1H), 3.05 (m, 1H), 2.66 (dd, 1H) and 1.28 (s, 9H).

### Example 8

### Enantiomer A of rel-(2R,4S,5R)-1-(3-Methoxy-4-tert-butylbenzoyl)-4-(pyrazin-2-yl)-2-(1,2-thiazol-3-ylmethyl)-5-(1,3-thiazol-2-yl)pyrrolidine-2-carboxylic acid

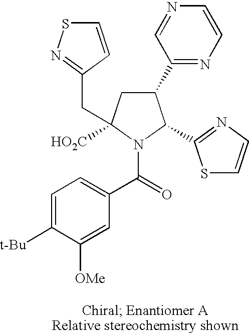

rel-(2R,4S,5R)-1-(3-Methoxy-4-tert-butylbenzoyl)-4-(pyrazin-2-yl)-2-(1,2-thiazol-3-ylmethyl)-5-(1,3-thiazol-2-yl)pyrrolidine-2-carboxylic acid (Example 7) was resolved by preparative chiral HPLC on a Chiralpak AD column using heptane-ethanol (70:30 v/v) containing 0.1% trifluoroacetic acid as eluent. Fractions containing the second eluting enantiomer were combined and evaporated. This crude product was dissolved in dichloromethane, washed with water; dried (hydrophobic frit) and solvent removed to give the title compound.

MS calcd for (C₂₈H₂₉N₅O₄S₂+H)⁺: 564

MS found (electrospray): (M+H)=564

¹H NMR (CD₃OD): δ 8.99 (d, 1H), 8.23 (d, 1H), 8.19 (d, 2H), 7.64 (d, 1H), 7.49 (d, 1H), 7.30 (d, 1H), 7.15 (d, 1H), 6.71 (dd, 1H), 6.67 (d, 1H), 5.59 (d, 1H), 4.16 (d, 1H), 3.72 (d, 1H), 3.71 (s, 3H), 3.27 (m, 1H), 3.05 (m, 1H), 2.66 (dd, 1H) and 1.29 (s, 9H).

### Example 9

### rel-(2R,4S,5R)-1-(3-Methoxy-4-tert-butylbenzoyl)-5-(5-methyl-1,3-thiazol-2-yl)-4-(pyrazin-2-yl)-2-(1H-pyrazol-1-ylmethyl)pyrrolidine-2-carboxylic acid

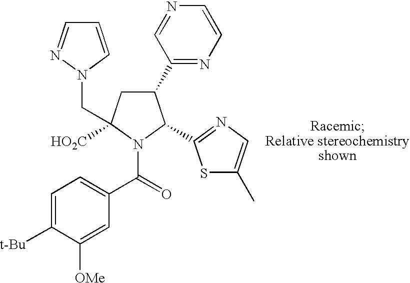

The title compound was prepared in a similar manner to Example 3, using Intermediate 22 in place of Intermediate 10.

MS calcd for (C₂₉H₃₂N₆O₄S+H)⁺: 561

MS found (electrospray): (M+H)⁺=561

¹H NMR (CDCl₃): δ 8.33 (1H, d), 8.27 (1H, bt), 8.13 (1H, bs), 7.67 (1H, d), 7.65 (1H, d), 7.35 (1H, d), 7.17 (1H, d), 6.72 (1H, dd), 6.46-6.43 (2H, m), 5.43 (1H, d), 5.28 (1H, d), 5.03 (1H, d), 3.63 (3H, s), 3.17 (1H, t), 2.97-2.88 (1H, m), 2.80 (1H, dd), 2.19 (3H, d) and 1.29 (9H, s). Acid proton not seen.

### Example 10

### Enantiomer A of rel-(2R,4S,5R)-1-(3-Methoxy-4-tert-butylbenzoyl)-5-(5-methyl-1,3-thiazol-2-yl)-4-(pyrazin-2-yl)-2-(1H-pyrazol-1-ylmethyl)pyrrolidine-2-carboxylic acid

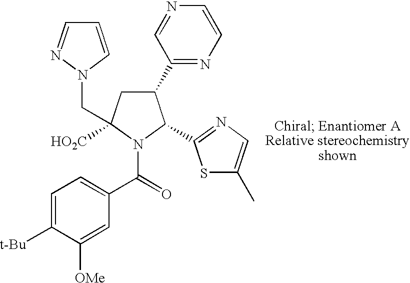

rel-(2R,4S,5R)-1-(3-Methoxy-4-tert-butylbenzoyl)-5-(5-methyl-1,3-thiazol-2-yl)-4-(pyrazin-2-yl)-2-(1H-pyrazol-1-ylmethyl)pyrrolidine-2-carboxylic acid (Example 9) was resolved by preparative chiral HPLC on a Chiralpak AD column using heptane-ethanol (70:30 v/v) containing 0.1% trifluoroacetic acid as eluent. Fractions containing the second eluting enantiomer were combined and evaporated. This crude product was dissolved in dichloromethane, washed with sodium hydrogen carbonate solution; dried (hydrophobic frit) and solvent removed to give the title compound.

MS calcd for (C₂₉H₃₂N₆O₄S+H)⁺: 561

MS found (electrospray): (M+H)⁺=561

¹H NMR (CDCl₃): δ 8.33 (1H, d), 8.27 (1H, t), 8.11 (1H, d), 7.66 (1H, d), 7.62 (1H, d), 7.35 (1H, bs), 7.17 (1H, d), 6.71 (1H, dd), 6.46-6.43 (2H, m), 5.43 (1H, d), 5.26-5.24 (1H, m), 5.03 (1H, m), 3.63 (3H, s), 3.21-3.10 (1H, m) 2.89-2.79 (2H, m), 2.19 (3H, s) and 1.29 (9H, s). Acid proton not seen.

### Example 11

### rel-(2R,4S,5R)-1-(3-Methoxy-4-tert-butylbenzoyl)-4-(pyrazin-2-yl)-2-(1H-pyrazol-1-ylmethyl)-5-(1,3-thiazol-2-yl)pyrrolidine-2-carboxylic acid

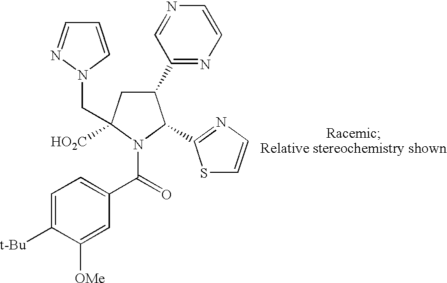

The crude product was obtained in a similar manner to Example 1, using Intermediate 24 in place of Intermediate 5, and was purified by chromatography on an SPE (NH₂) cartridge using methanol-formic acid (99:1 v/v) as eluent to give the title compound as a solid.

MS calcd for (C₂₈H₃₀N₆O₄S+H)⁺: 547

MS found (electrospray): (M+H)⁺=547

¹H NMR (CDCl₃): δ 14.85 (s, 1H), 8.31 (d, 1H), 8.22 (t, 1H), 8.12 (d, 1H), 7.74 (d, 1H), 7.69 (d, 1H), 7.64 (d, 1H), 7.15 (d, 1H), 7.06 (d, 1H), 6.70 (dd, 1H), 6.47 (d, 1H), 6.46 (t, 1H), 5.45 (d, 1H), 5.36 (d, 1H), 5.04 (d, 1H), 3.62 (s, 3H), 3.20 (t, 1H), 2.89 (m, 1H), 2.81 (dd, 1H) and 1.27 (s, 9H).

### Example 12

### Enantiomer A of rel-(2R,4S,5R)-1-(3-Methoxy-4-tert-butylbenzoyl)-4-(pyrazin-2-yl)-2-(1H-pyrazol-1-ylmethyl)-5-(1,3-thiazol-2-yl)pyrrolidine-2-carboxylic acid

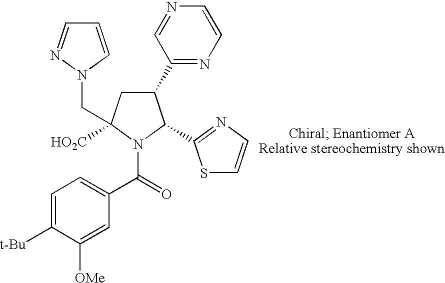

rel-(2R,4S,5R)-1-(3-Methoxy-4-tert-butylbenzoyl)-4-(pyrazin-2-yl)-2-(1H-pyrazol-1-ylmethyl)-5-(1,3-thiazol-2-yl)pyrrolidine-2-carboxylic acid (Example 11) was resolved by preparative chiral HPLC on a Chiralpak AD column using heptane-ethanol (80:20 v/v) containing 0.1% trifluoroacetic acid as eluent. The fractions containing the second eluting enantiomer were combined and evaporated. This crude product was dissolved in dichloromethane, washed with sodium carbonate solution; dried (hydrophobic frit) and solvent removed to give the title compound.

MS calcd for (C₂₈H₃₀N₆O₄S+H)⁺: 547

MS found (electrospray): (M+H)⁺=547

¹H NMR (d₆-DMSO): δ 8.17 (m, 2H), 7.91 (d, 1H), 7.87 (s, 1H), 7.64 (s, 1H), 7.35 (d, 1H), 7.06 (d, 1H), 6.98 (s, 1H), 6.74 (d, 1H), 6.54 (s, 1H), 6.39 (s, 1H), 5.26 (d, 1H), 5.09 (d, 1H), 4.87 (d, 1H), 3.51 (s, 3H), 3.02 (t, 1H), 2.31 (dd, 1H), 2.03 (m, 1H) and 1.24 (s, 9H).

### Example 13

### rel-(2R,4S,5R)-1-(3-Methoxy-4-tert-butylbenzoyl)-5-(5-methyl-1,3-thiazol-2-yl)-4-(pyrazin-2-yl)-2-(1,3-thiazol-2-ylmethyl)pyrrolidine-2-carboxylic acid

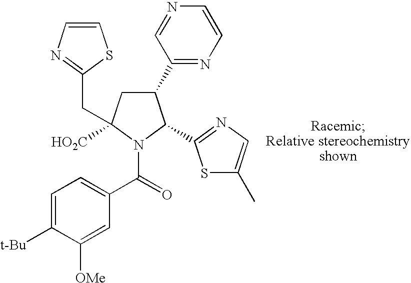

The crude product was obtained in a similar manner to Example 1, using Intermediate 27 in place of Intermediate 5, and was purified by column chromatography on silica gel eluting initially with cyclohexane-ethyl acetate (gradient elution from 50:1 v/v to 1:9 v/v), followed by dichloromethane:methanol (9:1 v/v) to give the title compound.

MS calcd for (C₂₉H₃₁N₅O₄S₂+H)⁺: 578

MS found (electrospray): (M+H)⁺=578

¹H NMR (CD₃OD): δ 8.29 (m, 1H), 8.22 (d, 2H), 7.90 (d, 1H), 7.69 (d, 1H), 7.36 (m, 1H), 7.20 (d, 1H), 6.87 (dd, 1H), 6.54 (d, 1H), 5.58 (d, 1H), 4.27 (d, 1H), 3.92 (d, 1H), 3.69 (s, 3H), 3.27 (m, 2H), 2.66 (m, 1H), 2.17 (d, 3H) and 1.29 (s, 9H).

### Example 14

### Enantiomer A of rel-(2R,4S,5R)-1-(3-Methoxy-4-tert-butylbenzoyl)-5-(5-methyl-1,3-thiazol-2-yl)-4-(pyrazin-2-yl)-2-(1,3-thiazol-2-ylmethyl)pyrrolidine-2-carboxylic acid

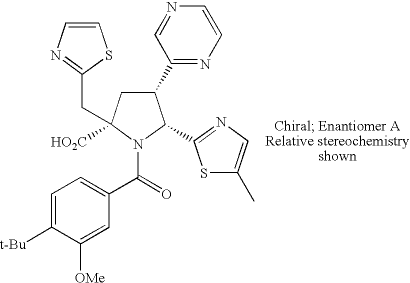

rel-(2R,4S,5R)-1-(3-Methoxy-4-tert-butylbenzoyl)-5-(5-methyl-1,3-thiazol-2-yl)-4-(pyrazin-2-yl)-2-(1,3-thiazol-2-ylmethyl)pyrrolidine-2-carboxylic acid (Example 13) was resolved by preparative chiral HPLC on a Chiralpak AD column using heptane-ethanol (70:30 v/v) containing 0.1% trifluoroacetic acid as eluent. The fractions containing the second eluting enantiomer were combined and evaporated. This crude product was dissolved in dichloromethane, washed with water; dried (hydrophobic frit) and solvent removed to give the title compound.

MS calcd for (C₂₉H₃₁N₅O₄S₂+H)⁺ 578

MS found (electrospray): (M+H)⁺=578

¹H NMR (CD₃OD): δ 8.31 (m, 1H), 8.24 (d 2H), 7.90 (d 1H), 7.69 (d 1H), 7.35 (m 1H), 7.20 (d 1H), 6.88 (dd 1H), 6.54 (d, 1H), 5.58 (d 1H), 4.29 (d 1H), 3.93 (d 1H), 3.68 (s 3H), 3.25 (m 2H), 2.68 (m 1H), 2.19 (d 3H), 1.30 (s 9H).

### Example 15

### rel-(2R,4S,5R)-1-(3-Methoxy-4-tert-butylbenzoyl)-5-(5-methyl-1,3-thiazol-2-yl)-4-(pyrazin-2-yl)-2-(1,3-thiazol-4-ylmethyl)pyrrolidine-2-carboxylic acid

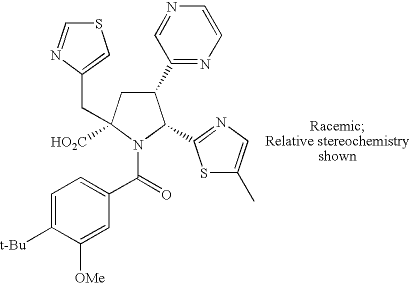

The title compound was prepared in a similar manner to Example 3, using Intermediate 30 in place of Intermediate 10.

MS calcd for (C₂₉H₃₁N₅O₄S₂+H)⁺: 578

MS found (electrospray): (M+H)⁺=578.

¹H NMR (CD₃OD): δ 9.15 (d, 1H), 8.28 (d, 1H), 8.25 (m, 1H), 8.20 (d, 1H), 7.59 (d, 1H), 7.37 (s, 1H), 7.20 (d, 1H), 6.82 (dd, 1H), 6.60 (d, 1H), 5.45 (d, 1H), 4.17 (d, 1H), 3.70 (s, 3H), 3.66 (d, 1H), 3.22 (d, 1H), 3.04 (m, 1H), 2.65 (dd, 1H), 2.18 (3, 3H) and 1.29 (s, 9H).

### Example 16

### Enantiomer A of rel-(2R,4S,5R)-1-(3-Methoxy-4-tert-butylbenzoyl)-5-(5-methyl-1,3-thiazol-2-yl)-4-(pyrazin-2-yl)-2-(1,3-thiazol-4-ylmethyl)pyrrolidine-2-carboxylic acid

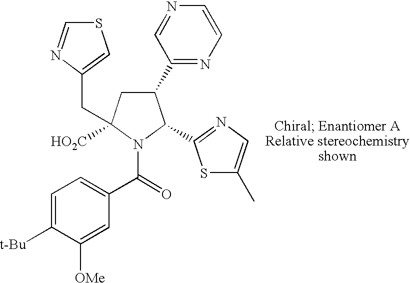

rel-(2R,4S,5R)-1-(3-Methoxy-4-tert-butylbenzoyl)-5-(5-methyl-1,3-thiazol-2-yl)-4-(pyrazin-2-yl)-2-(1,3-thiazol-4-ylmethyl)pyrrolidine-2-carboxylic (Example 15) was resolved by preparative chiral HPLC on a Chiralpak AD column using heptane-ethanol (70:30 v/v) containing 0.1% trifluoroacetic acid as eluent. Fractions containing the second eluting enantiomer were combined and evaporated. This crude product was triturated with ether to give the title compound.

MS calcd for (C₂₉H₃₁N₅O₄S₂+H)⁺: 578

MS found (electrospray): (M+H)⁺=578

¹H NMR (CD₃OD): δ 9.15 (d, 1H), 8.28 (d, 1H), 8.25 (m, 1H), 8.20 (d, 1H), 7.59 (d, 1H), 7.37 (s, 1H), 7.20 (d, 1H), 6.82 (dd, 1H), 6.60 (d, 1H), 5.45 (d, 1H), 4.17 (d, 1H), 3.70 (s, 3H), 3.66 (d, 1H), 3.22 (d, 1H), 3.04 (m, 1H), 2.65 (dd, 1H), 2.18 (3, 3H) and 1.29 (s, 91H).

### Example 17

### Enantiomer A of rel-(2R,4S,5R)-1-(3-Methoxy-4-tert-butylbenzoyl)-4-(pyrazin-2-yl)-2-(1,3-thiazol-4-ylmethyl)-5-(1,3-thiazol-2-yl)pyrrolidine-2-carboxylic acid

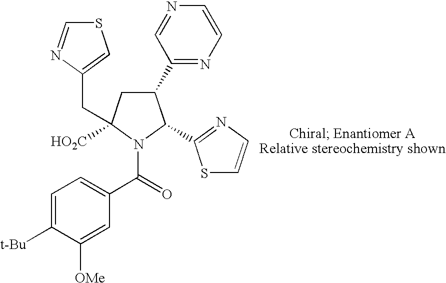

The crude product was obtained in a similar manner to Example 1, using Intermediate 33 in place of Intermediate 5, and was dissolved in dichloromethane and washed with water, dried (hydrophobic frit) and the solvent removed to give the title compound.

MS calcd for (C₂₈H₂₉N₅O₄S₂+H)⁺: 564

MS found (electrospray): (M+H)⁺=564

Analytical chiral HPLC on a Chiralpak AD column, using heptane-ethanol (60:40 v/v) containing 0.1% trifluoroacetic acid as eluent showed that the title compound was identical with Example 2.

¹H NMR (CDCl₃): δ 14.61 (s, 1H), 8.93 (d, 1H), 8.31 (d, 1H), 8.25 (dd, 1H), 8.08 (d, 1H), 7.73 (d, 1H), 7.36 (d, 1H), 7.13 (d, 1H), 7.04 (d, 1H), 6.67 (dd, 1H), 6.43 (d, 1H), 5.39 (d, 1H), 4.35 (d, 1H), 3.82 (d, 1H), 3.60 (s, 3H), 3.35 (m, 1H), 3.21 (t, 1H), 2.78 (dd, 1H) and 1.27 (s, 9H).

Preparation of Racemate of Example 19 from WO2003/037895A1

### Intermediate A1

### 2-[N-(1,3-Thiazol-2-ylmethylene)amino]-4-methylpentanolc acid, tert-butyl ester

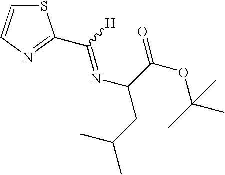

A stirred mixture of 2-amino-4-methyl-pentanoic acid tert-butyl ester, hydrochloride salt (5.00 g, 22.34 mmol), 1,3-thiazole-2-carboxaldehyde (2.53 g, 22.34 mmol) and triethylamine (3.10 mL, 22.3 mmol) in dichloromethane (60 mL) were heated under reflux under nitrogen for 19 hours. The reaction mixture was allowed to cool to room temperature, washed twice with water, dried over Na₂SO₄ and evaporated to give the title compound as an oil.

¹H NMR (CDCl₃): δ 8.46 (s, 1H), 7.94 (d, 1H), 7.44 (dd, 1H), 4.07 (dd, 1H), 1.89-1.74 (m, 2H), 1.64-1.52 (m, 1H), 1.48 (s, 9H), 0.96 (d, 3H) and 0.90 (d, 3H).

### Intermediate A2

### rel-(2S,4S,5R)-2-isobutyl-4-(pyrazin-2-yl)-5-(1,3-thiazol-2-yl) pyrrolidine-2-carboxylic acid, tert-butyl ester

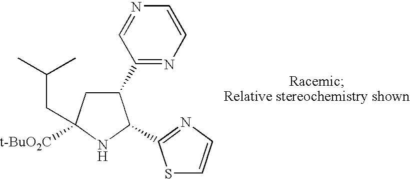

A mixture of 2-[N-(1,3-thiazol-2-ylmethylene)amino]4-methylpentanoic acid, tert-butyl ester (Intermediate A1; 1.0 g, 3.54 mmol), vinyl pyrazine (0.413 g, 3.9 mmol), lithium bromide (0.615 g, 7.08 mmol) and triethylamine (0.74 mL, 5.31 mmol) in tetrahydrofuran (15 mL) was stirred under nitrogen gas at 0° C. for 10 minutes then allowed to warm to room temperature overnight. A saturated solution of ammonium chloride was added and the mixture extracted with ethyl acetate (3×50 mL). The combined extracts were washed with water and brine, dried over sodium sulfate and evaporated. The residue was purified by chromatography on silica gel using cyclohexane-ethyl acetate (gradient elution from 50:1 v/v to 1:2 v/v) as eluent to give the title compound as an oil.

MS calcd for (C₂₀H₂₈N₄O₂S+H)⁺: 389

MS found:(M+H)⁺=389

### Intermediate A3

### rel-(2S,4S,5R)-2-Isobutyl-1-(3-methoxy-4-tert-butylbenzoyl) 4-(pyrazin-2-yl)-5-(1,3-thiazol-2-yl)pyrrolidine-2-carboxylic acid, tert-butyl ester

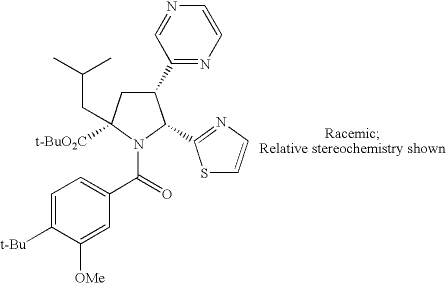

A mixture of rel-(2S,4S,5R)-2-Isobutyl-4-(pyrazin-2-yl)-5-(1,3-thiazol-2-yl)pyrrolidine-2-carboxylic acid, tert-butyl ester (Intermediate A2; 1.07 g, 2.75 mmol), 3-methoxy-4-tert-butylbenzoyl chloride (0.812 g, 3.58 mmol) and triethylamine (0.5 mL, 3.58 mmol) in dichloromethane (30 mL) was stirred at room temperature for 24 hours. The mixture was washed with water and the aqueous phase extracted with dichloromethane (2×20 mL). The combined organic solutions were washed with brine and dried over sodium sulfate and evaporated. The residue was purified by chromatography on silica gel using cyclohexane-ethyl acetate (gradient elution from 50:1 v/v to 3:2 v/v) as eluent to give the title compound as a solid.

MS calcd for (C₃₂H₄₂N₄O₄S+H)⁺: 579

MS found (electrospray): (M+H)⁺=579

### Example A

### rel-(2S,4S,5R)-2-Isobutyl-1-(3-methoxy-4-tert-butylbenzoyl)-4-(pyrazin-2-yl)-5-(1,3-thiazol-2-yl)pyrrolidine-2-carboxylic acid

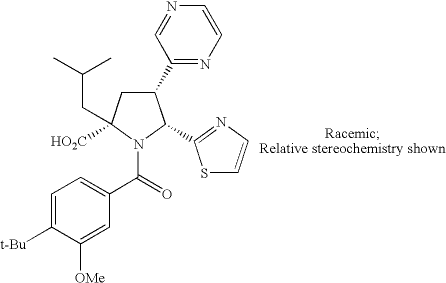

A solution of rel-(2S,4S,5R)-2-Isobutyl-1-(3-methoxy-4-tert-butylbenzoyl)-4-(pyrazin-2-yl)-5-(1,3-thiazol-2-yl)pyrrolidine-2-carboxylic acid, tert-butyl ester (Intermediate A3; 1.035 g, 1.79 mmol) in dichloromethane (10 mL) was treated with trifluoroacetic acid (15 mL) and the mixture stirred at room temperature for 5 hours. The mixture was evaporated to dryness and the residue purified by chromatography on silica gel using cyclohexane-ethyl acetate (gradient elution from 50:1 v/v to 1:4 v/v), then dichloromethane-methanol (gradient elution from 50:1 v/v to 19:1 v/v) as eluents to give the title compound as a solid.

MS calcd for (C₂₈H₃₄N₄O₄S+H)⁺: 523

MS found (electrospray): (M+H)⁺=523

¹H NMR (CD₃OD): δ 8.53 (d, 1H), 8.32 (d, 1H), 8.28 (dd, 1H), 7.66 (d, 1H), 7.33 (d, 1H), 7.19 (d, 1H), 6.76 (dd, 1H), 6.40 (d, 1H), 6.02 (d, 1H), 4.56 (m, 1H), 3.61 (s, 3H), 3.27 (t, 1H), 2.52 (dd, 1H), 2.35 (m, 2H), 2.17 (m, 1H), 1.28 (s, 9H), 1.20 (d, 3H) and 1.14 (d, 3H).

The chemical entities according to the invention may be formulated for administration in any convenient way, and the invention therefore also includes within its scope pharmaceutical compositions for use in therapy, comprising at least one chemical entity chosen from compounds of formula (Ia) and physiologically acceptable salts or solvates thereof in admixture with at least one physiologically acceptable diluent or carrier.

The chemical entities of the present invention can be administered by different routes including intravenous, intraperitoneal, subcutaneous, intramuscular, oral, topical, transdermal, or transmucosal administration. For systemic administration, oral administration is preferred. For oral administration, for example, the compounds can be formulated into conventional oral dosage forms such as capsules, tablets and liquid preparations such as syrups, elixirs and concentrated drops.

Alternatively, injection (parenteral administration) may be used, e.g., intramuscular, intravenous, intraperitoneal, and subcutaneous. For injection, the chemical entities of the invention are formulated in liquid solutions, preferably, in physiologically compatible buffers or solutions, such as saline solution, Hank's solution, or Ringer's solution. In addition, the chemical entities may be formulated in solid form and redissolved or suspended immediately prior to use. Lyophilized forms can also be produced.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays, rectal suppositories, or vaginal suppositories.

For topical administration, the chemical entities of the invention can be formulated into ointments, salves, gels, or creams, as is generally known in the art.

The amounts of various chemical entities to be administered can be determined by standard procedures taking into account factors such as the compound (IC₅₀) potency, (EC₅₀) efficacy, and the biological half-life (of the compound), the age, size and weight of the patient, and the disease or disorder associated with the patient. The importance of these and other factors to be considered are known to those of ordinary skill in the art.

Amounts administered also depend on the routes of administration and the degree of oral bioavailability. For example, for compounds with low oral bioavailability, relatively higher doses will have to be administered. Oral administration is a preferred method of administration of the present compounds.

Preferably the composition is in unit dosage form. For oral application, for example, a tablet, or capsule may be administered, for nasal application, a metered aerosol dose may be administered, for transdermal application, a topical formulation or patch may be administered and for transmucosal delivery, a buccal patch may be administered. In each case, dosing is such that the patient may administer a single dose.

Each dosage unit for oral administration contains suitably from 0.01 to 500 mg/Kg, and preferably from 0.1 to 50 mg/Kg, of a compound of Formula (Ia) or a pharmaceutically acceptable salt or solvate thereof, calculated as the free base. The daily dosage for parenteral, nasal, oral inhalation, transmucosal or transdermal routes contains suitably from 0.01 mg to 100 mg/Kg, of a compound of Formula (Ia). A topical formulation contains suitably 0.01 to 5.0% of a compound of Formula (Ia). The active ingredient may be administered from 1 to 6 times per day, preferably once, sufficient to exhibit the desired activity, as is readily apparent to one skilled in the art.

Compositions of Formula (Ia) and their pharmaceutically acceptable salts which are active when given orally can be formulated as syrups, tablets, capsules and lozenges. A syrup formulation will generally consist of a suspension or solution of the compound or salt in a liquid carrier for example, ethanol, peanut oil, olive oil, glycerine or water with a flavoring or coloring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule any pharmaceutical carrier routinely used for preparing dispersions or suspensions may be considered, for example aqueous gums, celluloses, silicates or oils, and are incorporated in a soft gelatin capsule shell.

Typical parenteral compositions consist of a solution or suspension of a compound or salt in a sterile aqueous or non-aqueous carrier optionally containing a parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional non-CFC propellant such as 1,1,1,2-tetrafluoroethane or 1,1,1,2,3,3,3-heptafluoropropane.

A typical suppository formulation comprises a compound of Formula (Ia) or a pharmaceutically acceptable salt thereof which is active when administered in this way, with a binding and/or lubricating agent, for example polymeric glycols, gelatins, cocoa-butter or other low melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

No unacceptable toxological effects are expected when compounds of the present invention are administered in accordance with the present invention.

### Assays

The potential for chemical entities of the invention to inhibit NS5B wildtype HCV polymerase activity, genotype 1a and genotype 1b, may be demonstrated, for example, using the following in vitro assays:

### In Vitro Detection of Inhibitors of HCV RNA-Dependent RNA Polymerase Activity

Incorporation of [³³P]-GMP into RNA was followed by absorption of the biotin labelled RNA polymer by streptavidin containing SPA beads. A synthetic template consisting of biotinylated 13mer-oligoG hybridised to polyrC was used as a homopolymer substrate.

a) Genotype 1a C-Terminally Truncated (delta21) Enzyme

HCV RNA Polymerase [Recombinant NS5B with C-terminal 21 amino acid deletion and C-terminal 6His-tag (Ferrari et al. J. Virol, 73(2), 1999, 1649. ‘Characterization of soluble hepatitis C virus RNA-dependent RNA polymerase expressed in Escherichia coli.’) expressed in E. coli and purified to homogeneity] was added to 25 nM final concentration. Polymerase of genotype 1a was from strain H77 (Yanagi, M., Purcell, R. H., Emerson, S. U. & Bukh, J. (1997), Proceedings of the National Academy of Sciences, USA 94, 8738-8743) containing a sequence change from valine to isoleucine at position 180.

Reaction Conditions were 25 nM enzyme, 1.5 μg/ml oligo-rG13/poly-rC and 0.2 μCi α-³³P-GTP in 0.5 μM GTP (20 Ci/mMol), 20 mM Tris pH 7.5, 23 mM NaCl, 3 mM DTT, 5 mM MgCl₂, 1 mM MnCl₂.

Enzyme was diluted to 500 nM concentration in 20 mM Tris-HCl, pH 7.5, 25 mM NaCl and 3 mM DTT.

4× concentrated assay buffer mix was prepared using 1M Tris-HCl, pH7.5 (1 mL), 5M NaCl (0.25 mL), 1M DTT (0.12 mL) and Water (8.63 mL), Total 10 mL.

2× concentrated first reagent was prepared using 4× concentrated assay buffer mix (5 μL), 40 u/μL RNasin (0.1 μL), 20 μg/mL polyrC/biotinylated-oligoG (1.6 μL), 500 nM enzyme (1 μL) and Water (2.3 μL), Total 10 μl/well.

2× concentrated second reagent was prepared using 1M MgCl₂ (0.1 μL), 1M MnCl₂ (0.02 μL), 25 μM GTP (0.4 μL), α-[³³P]-GTP (10 μCi/μL, 0.02 μL) and water (9.5 μL), Total 10 μL/well.

The assay was set up using compound (1 μL in 100% DMSO), first reagent (10 μL), and second reagent (10 μL), Total 21 μL.

The reaction was performed in a U-bottomed, white, 96-well plate. The reaction was mixed on a plate-shaker, after addition of the Enzyme, and incubated for 1 h at 22° C. After this time, the reaction was stopped by addition of 60 μL 1.5 mg/ml streptavidin SPA beads (Amersham) in 0.1 M EDTA in PBS. The beads were incubated with the reaction mixture for 1 h at 22° C. after which 100 μL 0.1 M EDTA in PBS was added. The plate was sealed, mixed centrifuged and incorporated radioactivity determined by counting in a Trilux (Wallac) or Topcount (Packard) Scintillation Counter.

After subtraction of background levels without enzyme, any reduction in the amount of radioactivity incorporated in the presence of a compound, compared to that in the absence, was taken as a measure of the level of inhibition. Ten concentrations of compounds were tested in three- or fivefold dilutions. From the counts per minute, percentage of inhibition at highest concentration tested or IC₅₀s for the compounds were calculated using GraFit 3, GraFit 4 or GraFit 5 (Erithacus Software Ltd.) software packages or a data evaluation macro for Excel based on XLFit Software (IDBS).

### b) Genotype 1b Full-Length Enzyme

Reaction Conditions were 0.5 μM [³³P]-GTP (20 Ci/mMol), 1 mM Dithiothreitol, 20 mM MgCl₂, 5 mM MnCl₂, 20 mM Tris-HCl, pH7.5, 1.6 μg/mL polyC/0.256 μM biotinylated oligoG13, 10% glycerol, 0.01% NP-40, 0.2 u/μL RNasin and 50 mM NaCl.

HCV RNA Polymerase (Recombinant full-length NS5B (Lohmann et al, J. Virol. 71 (11), 1997, 8416. ‘Biochemical properties of hepatitis C virus NS5B RNA-dependent RNA polymerase and identification of amino acid sequence motifs essential for enzymatic activity’) expressed in baculovirus and purified to homogeneity) was added to 4 nM final concentration.

5× concentrated assay buffer mix was prepared using 1M MnCl₂ (0.25 mL), glycerol (2.5 mL), 10% NP-40 (0.025 mL) and Water (7.225 mL), Total 10 mL.

2× concentrated enzyme buffer contained 1M-Tris-HCl, pH7.5 (0.4 mL), 5M NaCl (0.2 mL), 1M-MgCl₂ (0.4 mL), glycerol (1 mL), 10% NP-40 (10 μL), 1M DTT (20 μL) and water (7.97 mL), Total 10 mL.

Substrate Mix was prepared using 5× Concentrated assay Buffer mix (4 μL), [³³P]-GTP (10 μCi/μL, 0.02 μL), 25 μM GTP (0.4 μL), 40 u/μL RNasin (0.1 μL), 20 μg/mL polyrC/biotinylated-oligoG (1.6 μL), and Water (3.94 μL), Total 10 μL.

Enzyme Mix was prepared by adding 1 mg/ml full-length NS5B polymerase (1.5 μL) to 2.81 mL 2×-concentrated enzyme buffer.

The Assay was set up using compound (1 μL), Substrate Mix (10 μL), and Enzyme Mix (added last to start reaction) (10 μL), Total 21 μL.

The reaction was performed in a U-bottomed, white, 96-well plate. The reaction was mixed on a plate-shaker, after addition of the Enzyme, and incubated for 1 h at 22° C. After this time, the reaction was stopped by addition of 40 μL 1.875 mg/ml streptavidin SPA beads in 0.1 M EDTA. The beads were incubated with the reaction mixture for 1 h at 22° C. after which 120 μL 0.1 M EDTA in PBS was added. The plate was sealed, mixed centrifuged and incorporated radioactivity determined by counting in a Trilux (Wallac) or Topcount (Packard) Scintillation Counter.

After subtraction of background levels without enzyme, any reduction in the amount of radioactivity incorporated in the presence of a compound, compared to that in the absence, was taken as a measure of the level of inhibition. Ten concentrations of compounds were tested in three- or fivefold dilutions. From the counts, percentage of inhibition at highest concentration tested or IC₅₀s for the compounds were calculated using GraFit 3, GraFit 4 or GraFit 5 (Erithacus Software Ltd.) software packages or a data evaluation macro for Excel based on XLFit Software (IDBS).

The potential for compounds of the invention to inhibit NS5B wildtype HCV polymerase activity, genotype 1a and genotype 1b, may be demonstrated, for example, using the following cell based assays:

### Replicon ELISA Cell Based Assay

### Method

100 μL of medium containing 10% FCS were added to each well of clear, flat-bottomed 96 well microplates, excepting wells in the top row. Test compound was diluted in assay medium to twice the final required starting concentration from a 40 mM stock solution in DMSO. 200 μL of the starting dilution were introduced into two wells each in the top row and doubling dilutions made down the plate by the sequential transfer of 100 μL aliquots with thorough mixing in the wells; the final 100 μL were discarded. The two bottom rows were not used for compound dilutions. Huh-7 HCV replicon cell monolayers nearing confluency were stripped from growth flasks with versene-trypsin solution and the cells were resuspended in assay medium at either 2×10⁵ cells/mL (sub-line 5-15; genotype 1b; Lohmann, V., Komer, F., Koch, J-O., Herian, U., Thielmann, L. And Bartenschlager, R., 1999, Science, 285, pp 110-113) or at 3×10⁵ cells/mL (genotype 1a; Gu, B., Gates, A. T., Isken, O., Behrens, S. E. and Sarisky, R. T., J. Virol., 2003, 77, 5352-5359). 100 μL of cell suspension were added to all wells and the plates incubated at 37° C. for 72 hours in a 5% CO₂ atmosphere.

Following incubation, the assay medium was aspirated from the plates. The cell sheets were washed by gentle immersion in phosphate buffered saline (PBS), which was then aspirated off, and fixed with acetone:methanol (1:1) for 5 minutes. Following a further wash with PBS, 100 μL of ELISA diluent (PBS+0.05% v/v Tween 20+2% w/v skimmed milk powder) were added to all wells and the plates incubated at 37° C. for 30 minutes on an orbital platform. The diluent was removed and each well then received 50 μL of a 1/200 dilution of anti-HCV specific, murine, monoclonal antibody (either Virostat #1872 or #1877), except for wells in one of the compound-free control rows which received diluent alone to act as negative controls. The plates were incubated at 37° C. for 2 hours and washed 3 times with PBS/0.05% Tween 20, then 50 μL of horseradish peroxidase conjugated, anti-mouse, rabbit polyclonal serum (Dako #P0260), diluted 1/1000, were added to all wells. The plates were incubated for a further hour, the antibody removed and the cell sheets washed 5 times with PBS/Tween and blotted dry. The assay was developed by the addition of 50 μL of ortho-phenylenediamine/peroxidase substrate in urea/citrate buffer (SigmaFast, Sigma #P-9187) to each well, and colour allowed to develop for up to 15 minutes. The reaction was stopped by the addition of 25 μL per well of 2 M sulphuric acid and the plates were read at 490 nm on a Fluostar Optima spectrophotometer.

The substrate solution was removed and the plates were washed in tap water, blotted dry and the cells stained with 5% carbol fuchsin in water for 30 minutes. The stain was discarded and the cell sheets washed, dried and examined microscopically to assess cytotoxicity.

### Data Analysis

The absorbance values from all compound-free wells that had received both primary and secondary antibodies were averaged to obtain a positive control value. The mean absorbance value from the compound-free wells that had not received the primary antibody was used to provide the negative (background) control value. The readings from the duplicate wells at each compound concentration were averaged and, after the subtraction of the mean background from all values, were expressed as a percentage of the positive control signal. The quantifiable and specific reduction of expressed protein detected by the ELISA in the presence of a drug can be used as a measure of replicon inhibition. GraFit software was used to plot the curve of percentage inhibition against compound concentration and derive the 50% inhibitory concentration (IC₅₀) for the compound.

### Results

| | IC₅₀ | IC₅₀ in full | | |
|---|---|---|---|---|
| | in delta-21 | length 1b | IC₅₀ in 1a | IC₅₀ in 1b |
| | 1a enzyme | enzyme | replicon | replicon |
| | inhibition | inhibition | cell-based | cell-based |
| Compound | assay (μM) | assay (μM) | assay (μM) | assay (μM) |
| Example 1 | * | # | + | @ |
| Example 2 | * | # | + | @ |
| Example 3 | * | # | + | @ |
| Example 4 | * | # | + | @ |
| Example 5 | * | # | + | @ |
| Example 6 | * | # | + | @ |
| Example 7 | * | # | + | @ |
| Example 8 | * | # | + | @ |
| Example 9 | * | # | + | @ |
| Example 10 | * | # | + | @ |
| Example 11 | * | # | + | @ |
| Example 12 | * | # | + | @ |
| Example 13 | * | # | + | @ |
| Example 14 | * | # | + | @ |
| Example 15 | * | # | + | @ |
| Example 16 | * | # | + | @ |
| Compound A | *** | ## | ++ | @@ |
| Compound B | *** | ### | ++ | @@ |
| Compound C | ** | ## | ++ | @@ |
| Compound D | *** | ## | +++ | @@ |
| Compound E | *** | ## | ++ | @@ |
| Compound F | *** | ### | +++ | @@@ |
| Compound G | *** | ## | ++ | @@ |

Activity ranges

| | |
|---|---|
| Genotype 1a | Genotype 1b |

| | | | | | | |
|---|---|---|---|---|---|---|
| enzyme | * | <0.15 | μM | # | <0.10 | μM |
| | ** | 0.15-1.00 | μM | ## | 0.10-0.50 | μM |
| | *** | >1.00 | μM | ### | >0.50 | μM |
| replicon | + | <10.00 | μM | @ | <0.10 | μM |
| cell-based | ++ | 10.00-100 | μM | @@ | 0.10-10.00 | μM |
| | +++ | >100 | μM | @@@ | >10.00 | μM |

Compound A corresponds to the racemate of the enantiomeric compound disclosed as Example 19 in WO03/037895, rel-(2S,4S,5R)-2-Isobutyl-1-(4-tert-butyl-3-methoxybenzoyl)-4-(pyrazin-2-yl)-5-(1,3-thiazol-2-yl)pyrrolidine-2-carboxylic acid.

Compound B corresponds to the enantiomeric compound disclosed as Example 19 in WO03/037895, Enantiomer A of rel-(2S,4S,5R)-2-Isobutyl-1-(4-tert-butyl-3-methoxybenzoyl)-4-(pyrazin-2-yl)-5-(1,3-thiazol-2-yl)pyrrolidine-2-carboxylic acid.

Compound C corresponds to the racemic compound disclosed as Example 40 in WO03/037895, rel-(2R,4S,5R)-1-(4-tert-Butyl-3-methoxybenzoyl)-2-(pyridin-2-ylmethyl)-4-(pyrazin-2-yl)-5-(1,3-thiazol-2-yl)pyrrolidine-2-carboxylic acid.

Compound D corresponds to the racemic compound disclosed as Example 49 in WO03/037895, rel-(2R,4S,5R)-1-(4-tert-Butyl-3-methoxybenzoyl)-2-(1H-imidazol-4-yl-methyl)-4-(pyrazin-2-yl)-5-(1,3-thiazol-2-yl)-pyrrolidine-2-carboxylic acid.

Compound E corresponds to the racemic compound disclosed as Example 51 in WO03/037895, rel-(2R,4S,5R)-1-(4-tert-Butyl-3-methoxybenzoyl)-2-(2-(methylthio)ethyl)-4-(pyrazin-2-yl)-5-(1,3-thiazol-5-yl)-pyrrolidine-2-carboxylic acid.

Compound F corresponds to the racemic compound disclosed as Example 57 in WO03/037895, rel-(2R,4S,5R)-1-(4-tert-Butyl-3-methoxybenzoyl)-2-(2-(methylsulphonyl)ethyl)-4-pyrazin-2-yl-5-(1,3-thiazol-2-yl)-pyrrolidine-2-carboxylic acid.

Compound G corresponds to the racemic compound disclosed as Example 67 in WO03/037895, rel-(2R,4S,5R)-1-(4-tert-Butyl-3-methoxybenzoyl)-2-(phenylmethyl)-4-(pyrazin-2-yl)-5-(1,3-thiazol-2-yl)pyrrolidine-2-carboxylic acid.

Compound A may be prepared as described above in the Examples section as ‘preparation of racemate of example 19 from WO2003/037895A1’

Compounds B-G may be made according to the processes described in WO03/037895.

Structures of Compounds A-G are shown below for the avoidance of doubt.

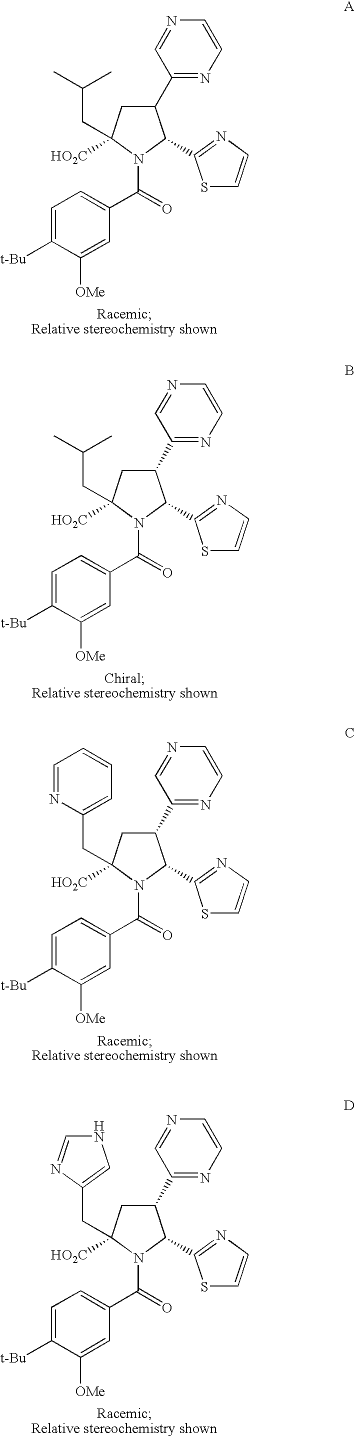

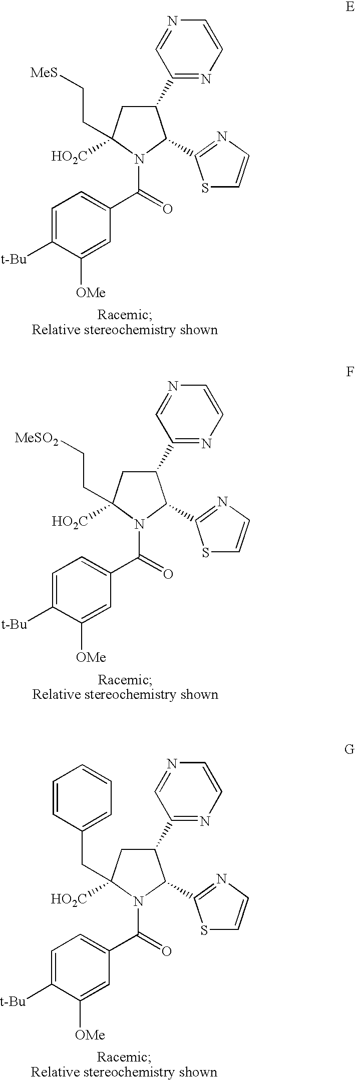

The compounds of the present invention which have been tested demonstrate a surprisingly superior genotype-1a/1a profile, as shown by the IC₅₀ values in the enzyme and cell-based assays across both of the 1a and 1a genotypes of HCV, compared to Compounds A-G. Accordingly, the compounds of the present invention are of great potential therapeutic benefit in the treatment and prophylaxis of HCV.

The pharmaceutical compositions according to the invention may also be used in combination with at least one other therapeutic agents, for example immune therapies (eg. interferon), therapeutic vaccines, antifibrotic agents, anti-inflammatory agents such as corticosteroids or NSAIDs, bronchodilators such as beta-2 adrenergic agonists and xanthines (e.g. theophylline), mucolytic agents, anti-muscarinics, anti-leukotrienes, inhibitors of cell adhesion (e.g. ICAM antagonists), anti-oxidants (eg N-acetylcysteine), cytokine agonists, cytokine antagonists, lung surfactants and/or antimicrobial and anti-viral agents (eg ribavirin and amantidine). The compositions according to the invention may also be used in combination with gene replacement therapy.

The invention thus provides, in a further aspect, a combination comprising at least one chemical entity chosen from compounds of formula (Ia) and physiologically acceptable salts or solvates thereof, together with at least one other therapeutically active agent.

The combinations referred to above may conveniently be presented for use in the form of a pharmaceutical formulation and thus pharmaceutical formulations comprising a combination as defined above together with at least one pharmaceutically acceptable diluent or carrier thereof represent a further aspect of the invention.

The individual components of such combinations may be administered either sequentially or simultaneously in separate or combined pharmaceutical formulations. Appropriate doses of known therapeutic agents will be readily appreciated by those skilled in the art.

All publications, including but not limited to patents and patent applications cited in this specification are herein incorporated by reference as if each individual publication were specifically and individually indicated to be incorporated by reference as though fully set forth.

What is claimed is:

1. At least one chemical entity chosen from compounds of Formula (Ia): 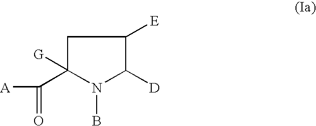
wherein
A represents hydroxy;
B represents C(O)R³ wherein R³ is 4-tert-butyl-3-methoxyphenyl;
D represents 1,3-thiazol-2-yl or 5-methyl-1,3-thiazol-2-yl;
E represents pyrazin-2-yl;
G represents 1,3-thiazol-2-ylmethyl, 1,3-thiazol-4-ylmethyl, 1,2-thiazol-3-ylmethyl, or 1H-pyrazol-1-ylmethyl;
and salts, solvates and esters thereof; provided that when A is esterified to form —OR where R is selected from straight or branched chain alkyl, aralkyl, aryloxyalkyl, or aryl, then R is other than tert-butyl.

2. At least one chemical entity chosen from compounds selected from the group consisting of:
rel-(2R,4S,5R)-1-(3-Methoxy-4-tert-butylbenzoyl)-4-(pyrazin-2-yl)-2-(1,3-thiazol-4-ylmethyl)-5-(1,3-thiazol-2-yl)pyrrolidine-2-carboxylic acid;
rel-(2R,4S,5R)-1-(3-Methoxy-4-tert-butylbenzoyl)-4-(pyrazin-2-yl)-2-(1,3-thiazol-2-ylmethyl)-5-(1,3-thiazol-2-yl)pyrrolidine-2-carboxylic acid;
rel-(2R,4S,5R)-1-(3-Methoxy-4-tert-butylbenzoyl)-5-(5-methyl-1,3-thiazol-2-yl)-4-(pyrazin-2-yl)-2-(1,2-thiazol-3-ylmethyl)pyrrolidine-2-carboxylic acid;
rel-(2R,4S,5R)-1-(3-Methoxy-4-tert-butylbenzoyl)-4-(pyrazin-2-yl)-2-(1,2-thiazol-3-ylmethyl)-5-(1,3-thiazol-2-yl)pyrrolidine-2-carboxylic acid;
rel-(2R,4S,5R)-1-(3-Methoxy-4-tert-butylbenzoyl)-5-(5-methyl-1,3-thiazol-2-yl)-4-(pyrazin-2-yl)-2-(1H-pyrazol-1-ylmethyl)pyrrolidine-2-carboxylic acid;
rel-(2R,4S,5R)-1-(3-Methoxy-4-tert-butylbenzoyl)-4-(pyrazin-2-yl)-2-(1H-pyrazol-1-ylmethyl)-5-(1,3-thiazol-2-yl)pyrrolidine-2-carboxylic acid;
rel-(2R,4S,5R)-1-(3-Methoxy-4-tert-butylbenzoyl)-5-(5-methyl-1,3-thiazol-2-yl)-4-(pyrazin-2-yl)-2-(1,3-thiazol-2-ylmethyl)pyrrolidine-2-carboxylic acid; and
rel-(2R,4S,5R)-1-(3-Methoxy-4-tert-butylbenzoyl)-5-(5-methyl-1,3-thiazol-2-yl)-4-(pyrazin-2-yl)-2-(1,3-thiazol-4-ylmethyl)pyrrolidine-2-carboxylic acid;
and salts, solvates and esters, and individual enantiomers thereof.

3. A method of treating or preventing viral infection which comprises administering to a subject in need thereof, an effective amount of at least one chemical entity chosen from compounds of Formula (Ia) and salts, solvates and esters thereof as claimed in claim 1.

4. A method as claimed in claim 3 wherein the viral infection is HCV.

5. A method as claimed in claim 3 in which the chemical entity is adminstered in an oral dosage form.

6. (canceled)

7. (canceled)

8. (canceled)

9. (canceled)

10. (canceled)

11. A pharmaceutical formulation comprising at least one chemical entity chosen from compounds of Formula (Ia) and salts, solvates and esters thereof as claimed in claim 1 in conjunction with at least one pharmaceutically acceptable diluent or carrier.

12. A process for the preparation of a compound of Formula (Ia) as defined in claim 1 comprising deprotection of a compound of Formula (II) 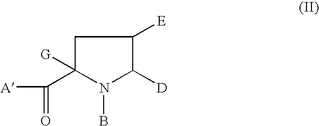
in which A′ is an alkoxy, benzyloxy or silyloxy group; B represents C(O)R³ wherein R³ is 4-tert-butyl-3-methoxyphenyl; D represents 1,3-thiazol-2-yl or 5-methyl-1,3-thiazol-2-yl; E represents pyrazin-2-yl; and G represents 1,3-thiazol-2-ylmethyl, 1,3-thiazol-4-ylmethyl, 1,2-thiazol-3-ylmethyl, or 1H-pyrazol-1-ylmethyl.
